SEQUENCE LISTING (12 sequences)

SEQ ID NO: 1 - DNA, 1155 bp, Homo sapiens
atgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatctttgttgcccgcctctactacttctgggacccagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactga

SEQ ID NO: 2 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 3 - protein, 195 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRH

SEQ ID NO: 4 - protein, 210 aa, Unknown
  mouse orthologue
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSS

SEQ ID NO: 5 - protein, 189 aa, Homo sapiens
SMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 6 - protein, 219 aa, Unknown
  mouse orthologue
TLSNICLTKGLPETRFCVEGRRVHLLSEEEFYSQFYNQRVKHLCYYHGMKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 7 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 8 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSISCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 9 - DNA, 730 bp, Homo sapiens
gccgccaccatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaa

SEQ ID NO: 10 - DNA, 730 bp, Homo sapiens; identical to SEQ ID NO: 9

SEQ ID NO: 11 - DNA, 21 bp, Unknown
  artificial organism, synthetic PCR oligonucleotide
tagacggcatcgcagcttgga

SEQ ID NO: 12 - DNA, 20 bp, Unknown
  artificial organism, synthetic PCR oligonucleotide
ctggtgatattgttgagtca

(19) United States
(12) Patent Application Publication
(10) Pub. No.: US 2004/0009951 A1
(43) Pub. Date: Jan. 15, 2004

(54) DNA deamination mediates innate immunity to (retro)viral infection

(76) Inventors: Michael H. Malim, London (GB); Ann M. Sheehy, London (GB); Reuben S. Harris, Cambridge (GB); Kate N. Bishop, London (GB); Michael S. Neuberger, Cambridge (GB); Nathan C. Gaddis, Emerald Isle, NC (US); James H.M. Simon, Rotterdam (NL)

Correspondence Address: Evelyn H. McConathy, Esquire, Dilworth Paxson LLP, 3200 Mellon Bank Center, 1735 Market Street, Philadelphia, PA, 19103, US

(21) Appl. No.: 10/460,923

(22) Filed: Jun. 13, 2003

Related U.S. Application Data
(60) Provisional application No. 60/388,513, filed on Jun. 13, 2002.
(60) Provisional application No. 60/472,952, filed on May 22, 2003.

(51) Int. Cl. A61K 48/00; C07H 21/02

(52) U.S. Cl. 514/44; 536/23.72

(57) ABSTRACT

Provided is a method of establishing an innate cellular immune defense mechanism against infectious (non-cellular) nucleic acid in a vertebrate or in vertebrate cells, comprising deaminating deoxycytidine (dC) to deoxyuridine (dU) in an infecting viral or retroviral nucleic acid in a dose-dependent manner by introducing a cellular nucleic acid deaminase. Also provided are such nucleic acid deaminases and the gene(s) encoding same, and uses therefore including thereapuetic formulations, methods of treatment, assays and methods of mutagenesis. Included are nucleic acid deaminases from the APOBEC family, in which CEM15 (also known as APOBEC3G) is one such nucleic acid deaminase.

### CROSS REFERENCE TO RELATED APPLICATIONS

This application claims priority to U.S. Provisional Application No. 60/388,513 filed Jun. 13, 2002, and to U.S. Provisional Application No. 60/472,952 filed May 23, 2003, the content of each of which is herein incorporated in its entirety.

### GOVERNMENT INTEREST

This invention was supported in part by Grants AI46246 and AI10460 from the National Institutes of Health, a grant from the Elizabeth Glaser Pediatric AIDS Foundation (40-01), and a Grant from the National Science Foundation. Accordingly, the Government may have certain rights in this invention.

### BACKGROUND OF THE INVENTION

Viruses have developed diverse innate/non-immune strategies to counteract host-mediated mechanisms that confer resistance to infection. Human immunodeficiency virus (HIV) is part of the Retroviridaie family, the members of which contain an RNA genome and reverse transcriptase activity. During their growth cycle, retroviruses copy their RNA into proviral DNA. The DNA is able to integrate into the chromosomal DNA of the host cell (called the provirus) where it uses the transcriptional and translational machinery of the host to express viral RNA and proteins. Viruses are released from the cell by budding from the cytoplasmic membrane. In the case of HIV-1 and HIV-2, viral replication results in the death of helper T-cell host cells, which leads to a state of severe immunodeficiency (acquired immune deficiency syndrome, AIDS), to the development of various malignancies and opportunistic infections, and ultimately to the death of the infected organism

The virus encoded virion infectivity factor (Vif) protein of HIV-1 is essential for the production of infectious virus by T lymphocytes and macrophages, the natural targets for HIV-1 infection. Vif is dispensable for HIV infection in certain transformed cell lines (“permissive” cells; (P)), but is required for propagative infection in others (“non-permissive” cells; (NP)) (Fisher et al., Science 237:888-893 (1987); Strebel et al., Nature 328:728-730 (1987)). The Vif protein has been shown to be required in virus producing cells, and in its absence infection is aborted at a post-entry step through the action of an innate anti-viral mechanism in human T lymphocytes, such that the infectivity of vif-deficient (Δvif) virions cannot be rescued by providing Vif in target cells (Gabuzda et al., J. Virol. 66:6489-6495 (1992); von Schwedler et al., J. Virol. 67:4945-4955 (1993); Sova et al., J. Virol. 67:6322-6326 (1993); Simon et al., J. Virol. 70:5297-5305 (1996); Strebel et al., 1987; Simon et al., EMBO J. 17:1259-1267 (1998A); Simon et al., Nature Med. 4:1397-1400 (1998B); Madani et al., J. Virol. 72:10251-10255 (1998)). Consequently, Vif proteins are essential for pathogenic infections in vivo (Fisher et al., 1987; Gabuzda et al., 1992; von Schwedler et al., 1993; Strebel et al., 1987; Desrosiers et al., J. Virol. 72:1431-1437(1998); Simon et al., 1998A; Simon et al., 1998B).

Vif is thought to act during virus assembly, budding and/or maturation; observations showing that Vif both associates with HIV-1 assembly intermediates and co-localizes with Gag in infected cells appear to support this view (Simon et al., J. Virol. 71:5259-5267 (1997); Simon et al., J. Virol. 73:2667-2674 (1999); Zimmerman et al., Nature 415:88-92 (2002)). Exposure of susceptible cells to Δvif viruses results in a lack of (or massive reduction in) provirus formation, with the defect being manifested after virus entry as an inability to accumulate reverse transcripts at normal levels (von Schwedler et al., 1993; Courcoul et al., J. Virol. 69:2068-2074 (1995); Simon et al., 1996; Sova et al., 1993).

Importantly, Δvif viruses only display their non-infectious phenotype (NP) when produced by primary human T cells, the principal cell target for HIV-1 in vivo, and a limited number of cell lines that includes HUT78 and CEM (Gabuzda et al., 1992; von Schwedler et al., 1993; Madani et al., 1998; Simon et al., 1998B). Conversely, many other cell lines such as SupT1, CEM-SS and 293T support the production of fully infectious Δvif virions (P). Cell fusion experiments have established that the NP phenotype is dominant over the P phenotype in that Δvif virions produced from NP×P heterokaryons are non-infectious (Madani et al., 1998; Simon et al., 1998B). In addition to regulating the infectivity of lentiviruses, earlier work has indicated that HIV Vif can also stimulate infection and replication of the distantly related onco-retrovirus MLV (Simon et al., 1998A). These findings supports a model in which NP cells selectively express an anti-viral activity that is suppressed by Vif, and predicts that NP cells express genes whose ectopic expression in P cells should phenotypically convert them to NP cells.

Current medical treatments for HIV infections include combinations of drugs that inhibit the action of the essential virus-encoding enzymes, reverse transcriptase (RT) and protease(PR). However, despite the tremendous benefits to quality of life and extended life span that have resulted from such treatment regimens, significant economical, compliance and drug-failure problems persist. Thus, in the absence of effective AIDS vaccines, the range of anti-HIV drugs needs to be expanded and improved. There still remains a need for more effective anti-viral therapies that are accompanied fewer side effects, e.g., little cellular toxicity and reduced immuno-stimulatory response.

### SUMMARY OF THE INVENTION

A novel protein, CEM15 (also known as APOBEC3G), a member of the family of cellular nucleic acid deaminases, is provided, which is a cellular protein that is packaged into virus particles. CEM15 is central to the blockade of viral infection at a post-entry step in the absence of the Vif protein (also reported by the inventors at Sheehy et al., Nature (2002)). CEM15 is a member of the APOBEC family of proteins whose founder (APOBEC1) edits RNA (Teng et al, Science 260:1816-1819 (1993); Jarmuz et al., Genomics 79:285-296 (2002)). However, genetic evidence (also reported by the inventors at Harris et al., Mol. Cell 10:1247-1253 (2002)) indicates that CEM15/APOBEC3G and related genes are able to mutate DNA by deaminating deoxycytidine (dC) to deoxyuridine (dU), thereby forming an innate defense mechanism against (retro)viral infection and providing for the first time a new strategy for inhibiting viral infection. Until the present invention there has not been biochemical evidence or disclosure of this innate defense (see also Harris et al., Nat. Immunol. (2003 in press)). For the purposes of this invention, however, the novel protein will be referred to simply as “CEM15” without the alternative name “APOBEC3G,” but such alternative name will be understood.

Also provided in the present invention are two other identified novel genes encoding CEM226 and CHIP290, which although apparently unrelated to Vif action, may also provide approaches for inhibiting HIV-1 infection when their normal functions are manipulated, alone or in conjunction with treatments involving CEM15.

Using a replication-defective murine leukemia virus (MLV) system, the present invention shows that CEM15 expressed in virus-producer cells induces a striking accumulation of G->A transitions in the plus-strands of retroviral cDNAs isolated from target cells. Such mutations cannot be explained by C->U transitions in virus genomic RNA, but rather indicate massive dC->dU deamination of minus (first)-strand cDNA. These findings imply that DNA deamination underlies a major strategy of immunity to retroviruses and may also contribute to HIV sequence variation.

The advantage of utilizing CEM15, CEM226 and CHIP290 as potential drug targets for HIV therapeutics is their novelty. In particular, drugs such as bioavailable small molecules that impede Vif's ability to block CEM15 function (or conversely enhance CEM15 activity, i.e., also see Harris et al., Nat. Immunol. (2003 in press)) will reduce HIV replication, infection and associated disease conditions. By exploiting aspects of virus replication that are unrelated to RT or PR function, it may be possible to expand and extend the breadth of anti-HIV strategies. Such an approach is highly advantageous as complications related to drug resistance continue to escalate.

In yet another aspect, the invention provides pharmaceutical formulations suitable for inhibiting and treating viral infection, particularly retroviral infection, specifically, although not limited to, HIV-1 infection, and having reduced side effects, such as immunogenicity. These formulations for inhibiting comprising at least one CEM15 expressing cell in accordance with the invention in a pharmaceutically acceptable carrier. In such formulations, CEM15 is preferably administered to the patient as expressed from a “CEM15 expressing cell” in an amount effective to inhibit the proliferation of the virus. Thus, the expression of the formulation occurs from “CEM15 in a virus-producer cell.” Because infectivity is restored in the presence of HIV Vif as disclosed, the preferred administration of CEM15 is in “Vif-resistant forms of CEM15.” The term CEM15 expressing cell, as it is used throughout the disclosure, is preferably intended to include Vif resistant forms of CEM15, but may also include over-expression resulting in dominance (by sheer abundance) over Vif action.

Further provided in another aspect of the invention are cell lines lacking in CEM15 expression. Such cell lines may be produced either by “knocking out” DNA deaminases or by “knocking in” HIV Vif, that would be suitable for production of retroviral vectors for gene therapy. The invention also facilitates the identification of cell lines that do not express APOBEC family member proteins—these cell lines may therefore be better suited than existing cell lines as gene therapy materials by removing any possibility of vector mutation/destruction triggered by deamination. Moreover, in light of the present disclosure, it is now makes it possible to create mutants, evolve proteins, and optimize protein expression (codon optimization) amongst other applications via mutagenesis of a retroviral-encoded cDNA (simply by growing it on a CEM15 expressing cell). This aspect of the invention is particularly useful when coupled to selections/screens. Accordingly, the invention is intended to encompass such mutants, proteins, protein expression, as well as methods of selection and screening. In other words, anything encoded in the retrovirus which, after growth in the CEM15 expressing cell, can then be selected/screened for in the target cell, making technologies incorporating enhanced/altered intrinsic protein fluorescence (GFP⁺), enhanced catalysis, altered substrate specificity, modified antibody affinity or alteration of any gene, genetic element or protein attribute especially powerful.

In another aspect, the invention provides a method of treating HIV-1 infection in a mammalian patient using the formulations described above. For purposes of the invention, the term “mammal” is meant to encompass primates and humans, although recognized laboratory animal equivalents are also included for experimental purposes.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE DRAWINGS

The foregoing summary, as well as the following detailed description of the invention, will be better understood when read in conjunction with the appended drawings. It should be understood, however, that the invention is not limited to the precise arrangements and instrumentalities shown.

FIG. 1 shows replication of HIV-1 in non-permissive (NP) CEM and permissive (P) CEM-SS T cells challenged with normalized stocks of wild type or Δvif X4-tropic viruses. Virus replication was monitored as the supernatant accumulation of p24^{Gag}. Solid squares=wild type virus; open circles=Δvif virus.

FIG. 2 is a Northern blot analysis of CEM15 expression in NP and P cells. RNAs extracted from the indicated panel of infected or uninfected cells were resolved by electrophoresis, and probed with ³²P-labeled CEM15 (upper panel). Following autoradiography, the filter was stripped and rehybridized with a β-actin specific probe to establish equivalent loading (lower panel).

FIGS. 3A-3D show that expression of CEM15 in permissive cells inhibits the infectivity of HIV-1/Δvif. 293T monolayers were transfected with cocktails comprising provirus (wild type or Δvif), pcDNA3.1-based vector (titration of pCEM15:HA with the empty parental vector making up the balance), and pLUC and varying amounts of pCEM15:HA to achieve the indicated ratios of CEM15:provirus. FIG. 3A shows an analysis of virus output from post-transfection virus-containing supernatants, wherein virus production is quantified in ng/ml p24^{Gag} by ELISA. FIG. 3A presents the mean values and standard deviations for eight independent experiments. FIG. 3B shows the effect of single-round virus infectivity using the viruses from FIG. 3A, corresponding to 5 ng p24 ^{Gag}, to challenge of C8166-CCR5/HIV-CAT cells. Productive infection was measured as the induction of CAT activity and values are presented as % infectivity relative to virus-alone samples (i.e., no pCEM15:HA used). FIG. 3B data were also derived from eight experiments. FIG. 3C shows protein expression of CEM15:HA as confirmed for one series of Δvif transfections by immunoblot analysis of whole cell lysates using a HA-specific monoclonal antibody. FIG. 3D shows luciferase expression of the cell lysates used for FIG. 3C. Values are displayed as % activity relative to the virus-alone sample.

FIGS. 4A-4B show that stable expression of CEM15 in CEM-SS cells selectively inhibits HIV-1/Δvif replication. FIG. 4A shows the transduction of CEM-SS cell cultures transduced with the NG/neo (negative control) or NG/C15 retroviral vectors, maintained in selective medium, and subjected to immunoblotting using antibodies specific for hnRNP C½ (loading control) or the HA epitope tag. FIG. 4B shows the replication of HIV-1, wherein control or CEM15 expressing cultures were challenged with wild type or Δvif virus corresponding to 1 ng p24^{Gag} and monitored as in FIG. 1. Open squares=control cells and wild type virus; solid closed squares=control cells and Δvif virus; open circles=CEM15 expressing cells and wild type virus; solid closed circles=CEM15 expressing cells and Δvif virus.

FIGS. 5A-5C provide the nucleotide and amino acid sequences of CEM15 and comparisons of the polypeptide with other polypeptides. FIG. 5A provides the nucleic acid sequence for the CEM15 gene (SEQID NO:1) and FIG. 5B provides the corresponding amino acid sequence of CEM15 (SEQID NO:2). FIG. 5C is a ClustalW alignment showing similarity of the amino-terminal domain of CEM15 (AAG14956) (SEQID NO:3), and carboxyl-terminal domain of CEM15 (AAG14956) (SEQID NO:4), its mouse orthologue (AAH03314) amino terminal domain (SEQID NO:5) and carboxyl terminal domain (SEQID NO:6), human phorbolin-1 (AAA03706) (SEQID NO:7) and human Apobec1 (NP_{—}001635) (SEQID NO:8). Residue numbers for each line of sequence are provided. Symbols asterisk (*), colon (:), and period (.) indicate identical, conserved or semi-conserved residues, respectively. The boxed region indicates the Prosite cytosine nucleoside/nucleotide deaminase zinc-binding motif PS00903, with putative zinc binding histidine and cysteine residues shown in bold-faced type.

FIGS. 6A-6B show the modulation of MLV infection and DNA deamination by CEM15. FIG. 6A graphically depicts the proportion of YFP-positive cells ˜48 hours after they were produced by co-transfection with 293T-derived stocks of YFP-encoding MLV using either a control vector or amounts of pCEM15:HA to challenge murine fibroblasts across a range of input inocula (normalized units of RT). FIG. 6B is a gel showing the effect of deamination of dC within a single-stranded oligonucleotide by purified His-tagged CEM15, as well as by recombinant APOBEC1 control, as monitored by subsequent treatment of the oligonucleotide with uracil-DNA glycosylase (UDG)/NaOH which breaks the oligonucleotide at the site of dC->dU deamination.

FIGS. 7A-7C show mutation of retroviral DNA by CEM15. FIG. 7A diagrammatically shows the percentages of target cells after challenge with MLV-GFP virions that were derived from either 293T cells stably expressing CEM15 or a control plasmid. FIG. 7B provides profiles of the relative positions of the G->A transition mutations apparent in 12 representative 730 bp GFP sequences derived from GFP-lo and GFP-hi populations of cells challenged with MLV-GFP grown on either CEM15-expressing cells or non-expressing controls. Two independently derived CEM15+clones (and two independent controls) were analyzed for each experiment (separated by a space). The G->A transitions are depicted by vertical lines; whereas other single nucleotide substitutions (20 in total) are indicated by lollipops. Thick, horizontal black bars represent the two deletions detected. FIG. 7C provides a comparison of the extent of GFP mutation in the different samples. The pie charts depict the proportion of MLV sequences carrying the indicated number of mutations within the sequenced 730 bp interval. The total number of sequences determined in each data set is indicated in the center.

FIGS. 8A-8D depict retroviral cDNA mutation spectra caused by CEM15-expression. FIG. 8A depicts the mutations detected among the GFP sequences amplified from GFP-lo enriched target cell populations (SEQID NO: 9). Mutations derived from MLV-GFP challenged target cells in which the viral stocks used were grown in CEM15-expressing cells are depicted above the 730 bp consensus (the viral plus or coding strand is shown) and those derived from vector-expressing cells are shown below. FIG. 8B depicts GFP mutations from GFP-hi enriched cell populations (SEQID NO:10). FIG. 8C diagrammatically depicts nucleotide substitution preferences for the entire set of GFP mutations detected in target cells infected with MLV-GFP that had been grown on a CEM15-expressing producer cells, in comparison to those detected in target cells infected with MLV-GFP grown on a vector-expressing producer cells. FIG. 8D diagrammatically delineates the preferred local sequence context for CEM15-mediated dC deamination in MLV-GFP.

FIGS. 9A-9C show that CEM15 is packaged into MLV particles and its function is inhibited by HIV Vif. FIG. 9A is a gel showing the YFP-encoding MLV virions that were purified from cells as described in FIG. 6A that either did not (lane 1) or did express CEM15 (lane 2), as analyzed by immunoblotting using antibodies specific for MLV Gag or CEM15. FIG. 9B diagrammatically shows that HIV Vif diminishes CEM15-mediated immunity to YFP-encoding MLV as monitored using the transient expression system (as in FIG. 6) in the presence of pCEM15:HA, with or without pcVIF. FIG. 9C shows the restoration of infectivity of CEM15-exposed MLV-GFP by expression of HIV Vif during viral stock production from 293T cells stably expressing CEM15.

### DETAILED DESCRIPTION OF PREFERRED EMBODIMENTS OF THE INVENTION

It is characteristic of human immunodeficiency viruses that they exhibit a high degree of variability, which significantly complicates the comparability of the different isolates. For example, when diverse HIV-1 isolates are compared, high degrees of variability are found in some regions of the genome while, other regions are comparatively well conserved (Benn et al., Science 230:949-951 (1985)). HIV-1 encodes nine genes; the gag, pol and env genes are common to all retroviruses, whereas the other six are restricted to the lentivirus sub-family. The greatest degree of genetic stability is possessed by regions in the gag and pol genes, which encode proteins that are essential for structural and enzymatic purposes. Some regions in the env gene, and the genes (vif, vpr, tat, rev and nef) encoding regulatory proteins, exhibit a high degree of variability.

These additional genes are often referred to as the regulatory or accessory genes, and subserve a variety of diverse functions during virus replication. Of particular interest to the present invention has been the vif gene, which is known to be a potent regular of virus infectivity—hence the derivation of its name, Viral Infectivity Factor. In tissue culture models, Vif can be absolutely required for HIV-1 replication, with the magnitude of the effect on a single cycle of infection being ˜100-fold. Complementing such findings, work with the related simian virus SIV_{MAC} has shown that Vif is essential for pathogenic infections in an appropriate monkey host.

Although the precise mechanism by which Vif imparts infectivity on HIV-1 particles remains unknown, and it is unclear whether Vif action results in significant protein, nucleic acid, or lipid differences in virions that relate to the infectivity effect, there is general agreement in the art that vif-deficient (Δvif) HIV-1 infections are defective at a step of the life cycle that follows entry into susceptible cells. In that case, the sustained accumulation of reverse transcripts and provirus formation both fail to occur.

For the following reasons, Vif function appeared to be intimately linked to the activity of host cell factors/genes, and therefore, the identification of such factors was pursued in the present invention. First, Vif function is restricted in a species-specific manner; that is to say that Vif proteins from divergent simian SIV lineages fail to function in human cells, but they can act in cells of the cognate host monkey species. The exceptions to this finding are the Vif proteins encoded by SIVs that naturally infect chimpanzees or sooty mangabeys. These do function in human cells, and these SIVs have crossed into human populations and become established as HIV-1 and HIV type-2, respectively, making Vif an important contributing factor to primate lentivirus zoonoses. Second, whereas Vif is required for HIV-1 infection in primary human T cells and certain cell lines, such as H9 and CEM (known as non-permissive, or NP, cells due to the restriction to Δvif virus growth), it is entirely dispensable in other lines such as CEM-SS and 293T (known as permissive, or P, cells). As noted above, to establish the basis for this differential Vif requirement for HIV-1 infectivity, classical cell fusion experiments have been used to demonstrate that the NP cell phenotype is dominant over the P phenotype. This finding implies that NP cells express factor(s) that inhibit HIV-1/Δvif infectivity, and that the role of Vif is, therefore, to overcome this suppression. The present invention was designed to identify such genes.

Based on the reasoning outlined above, the inventors determined that genes (and hence mRNAs) related to Vif function would be expressed in NP cells, but not in P cells. To identify such genes, and to evaluate their possible impact on HIV-1 infection and replication, the following experimental design was developed in terms of a number of discrete phases, as briefly summarized below:

i) Messenger RNA (mRNA) was isolated from one NP line, CEM, and one P line, CEM-SS. These lines represented a sensible choice since their relatedness, yet opposite phenotype with respect to Vif function, likely minimized the differences in MRNA expression profiles, thus reducing the “noise” in the study.

ii) Complementary DNA (cDNA) pools were generated by standard methods and subjected to polymerase chain reaction (PCR)-based subtraction (PCR-Select, Stratagene, La Jolla, Calif.) followed by cloning and DNA sequencing, or cDNA microarray hybridization (Incyte Genomics, Palo Alto, Calif.), for which additional detail will be provided below. Using this process, it is possible to amplify DNA sequences if regions of the sequence to be amplified are known. In each case, the CEM-SS sample was used as the “driver” so that identification of mRNAs selectively present in the CEM sample would be favored. Results from the two screens were combined and candidate genes (especially those recognized in both systems) were analyzed further.

iii) Northern analyses were used to identify genes preferentially (or exclusively) expressed in a panel of NP cells (CEM, H9, HUT78), but not P cells (CEM-SS and SupTI). This step helped to eliminate background genes that are expressed in CEM cells, but which are unrelated to NP activity.

iv) Full length cDNAs were derived for candidate genes and “epitope-tagged” expression vectors were created.

v) 293T cells (a P cell line) were transiently co-transfected with wild type or Δvif HIV-1 proviral expression vectors together with increasing levels of the candidate cDNA and an internal control plasmid expressing luciferase. At 24 to 48 hours post-transfection, the levels of virus in the culture supernatants were monitored by ELISA for p24^{Gag} expression to evaluate their effects on HIV-1 production. These viral stocks were normalized according to p24^{Gag} content and used in challenges of an indicator cell line in which productive infection is registered as expression of the reporter enzyme, chloramphenicol acetyl transferase (CAT).

vi) As a final analysis, 293T cell lysates from step v) were prepared and assessed for luciferase expression to rule out possible “genotoxic” effects of transfected cDNAs. Based on the previous description of Vif function, the gene(s) of interest were expected to have no significant impact on virus production. However, it was important that the gene(s) had to inhibit the infectivity of a Δvif virus in a dose-dependent fashion—while at the same time not affecting (or minimally affecting) the wild type viral counterpart.

vii) Any gene or genes that fulfilled these foregoing criteria were then stably expressed in CEM-SS cells (a P cell line) using a standard retroviral vector gene delivery system, and the consequences for spreading infections of wild type and Δvif virus were determined.

As a result of the foregoing strategy, an amalgamation of the two screens identified ˜200 candidate genes for further study. Northern analyses of RNAs isolated from a panel of NP and P cells were performed. Approximately ten of the candidate genes were found to have similar expression profiles; that is, clear expression in NP cells with no (or low) expression in P cells. This panel of candidate genes including expression of mRNA from a clone, called CEM15 by the inventors, that precisely matched the profile sought. Full-length cDNAs were derived for these genes, and they were “epitope-tagged” with the well-characterized antigenic octapeptide sequence derived from influenza virus hemagglutinin (recognized by the 12CA5 monoclonal antibody). These expression vectors were co-transfected into 293T cells with wild type or Δvif HIV-1 proviruses as a titration and virus production and infectivity determined. As a result, three candidate genes, CEM15, CEM226 and CHIP290, were selected from the original 200.

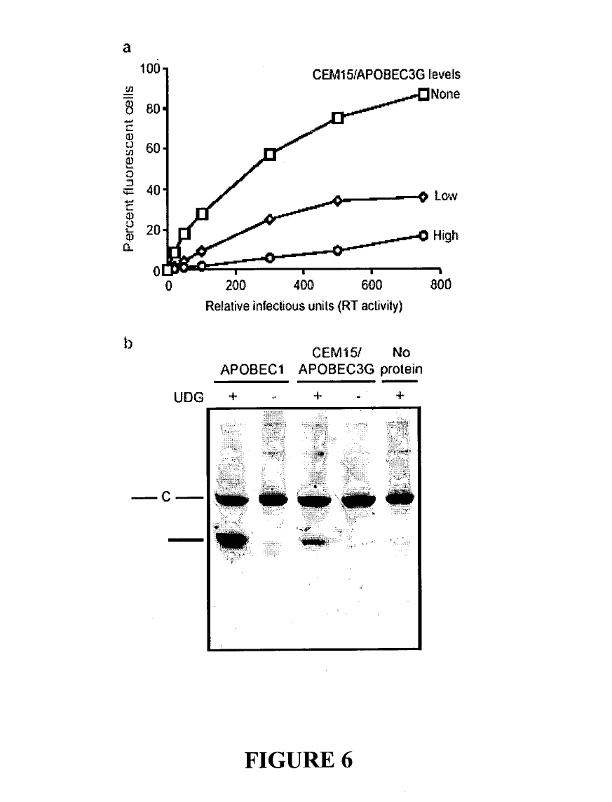

CEM15. The accession number for this gene is AF182420; it has also been called APOBEC3G (accession number NM_{—}021822), phorbolin like protein MDS019 (accession number BC024268) and MDS019. At the time of discovery, there were no reports in the published literature describing the normal function for this protein, though the most similar genes (by protein sequence) are phorbolin (also of unknown function) and the catalytic subunit of apolipoprotein B mRNA editing enzyme, apobec-1. As shown in the present invention, although not intending to be so limiting, CEM15 works by being carried into newly infected cells by virions and then deaminating retroviral DNA, leading to hypermutation, DNA destruction and/or DNA synthesis defects (FIGS. 6 to 9).

CEM226. This is a previously reported interferon-induced gene termed 1-8D (Lewin et al, Europ. J Biochem. 199:417-423 (1991); also known as IFITM2). CEM226 is one of a three-member family of related genes that also includes 9-27 and 1-8U. Importantly, the inventors have shown that 9-27 and 1-8U do not display the pronounced anti-HIV-1 effect in assays of the type described here, which helps to underscore the specificity of the 1-8D phenotype. The normal function of 1-8D has not been described, though the anti-viral effects of the interferons and their downstream targets are well known.

CHIP290. This is a previously described (U.S. Pat. No. 6,204,374), seven transmembrane G-protein coupled receptor (GPR), also called T cell death associated gene (TDAG8). Other than a proposed role in negative selection of self-reactive thymocytes in a murine model, no function has been ascribed to TDAG8.

In terms of virus production, CEM15 had no significant effect, while CEM226 and CHIP290 had profound inhibitory effects on both wild type and Δvif viruses. For progeny virion infectivity, CEM226 and CHIP290 were dose-dependent inhibitors and CEM15 was a potent repressor of vif-deficient infection. In all cases, luciferase expression was relatively constant across the titrations of the candidate genes, indicative of a lack of pleiotropic toxicity.

Thus, it was determined the CEM15, in particular, has the characteristics predicted for a gene involved in rendering NP cells refractive to HIV-1/Δvif growth. This was confirmed in a second experimental configuration where CEM-SS cells (a P cell line) were modified to express CEM15 constitutively. Unlike control cells, which support wild type and Δvif virus replication equivalently, the CEM-SS/CEM15 cells severely restricted Δvif virus replication. The residual viral growth that was noted was likely due to the presence of cells in the culture that did not express CEM15 at sufficient levels.

Accordingly, CEM15, CEM226 and CHIP290 each have the potential to provide novel anti-viral strategies. For CEM15, one approach would be to interfere with the ability of Vif to suppress the function of CEM15. Because CEM226 and CHIP290 are inhibitors of wild type HIV-1, and are expressed in primary T lymphocytes, one would be seeking to enhance this phenotype in HIV infected (or exposed) cells. This could be via stimulation of function, up-regulation of expression or the removal of natural counteracting inhibitors. For example, since CHIP290 is a G-protein coupled receptor, the stimulation of action through the use of pharmacological agonists is feasible. Finally, the anti-viral nature of these genes (or modified versions thereof) make them candidates for possible gene therapy strategies (e.g., over expression of, or administration of, Vif-resistant forms of CEM15 would be predicted to inhibit virus replication).

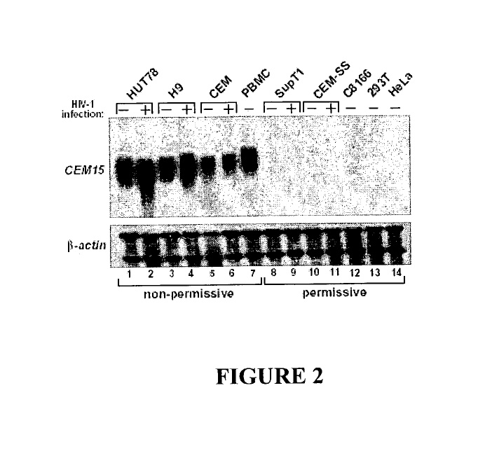

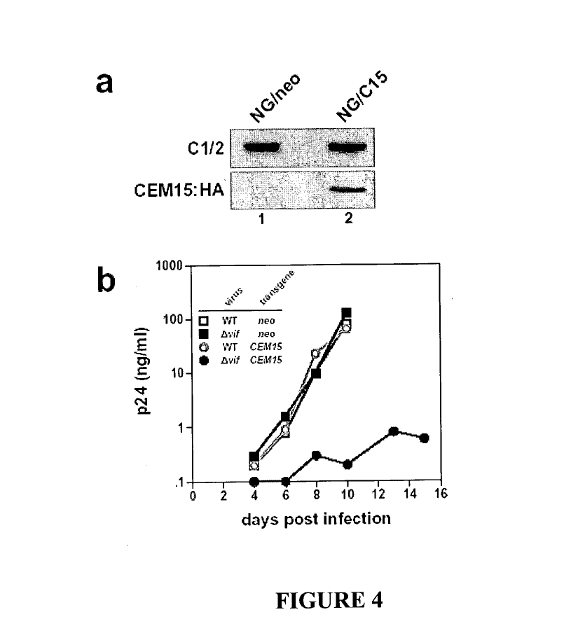

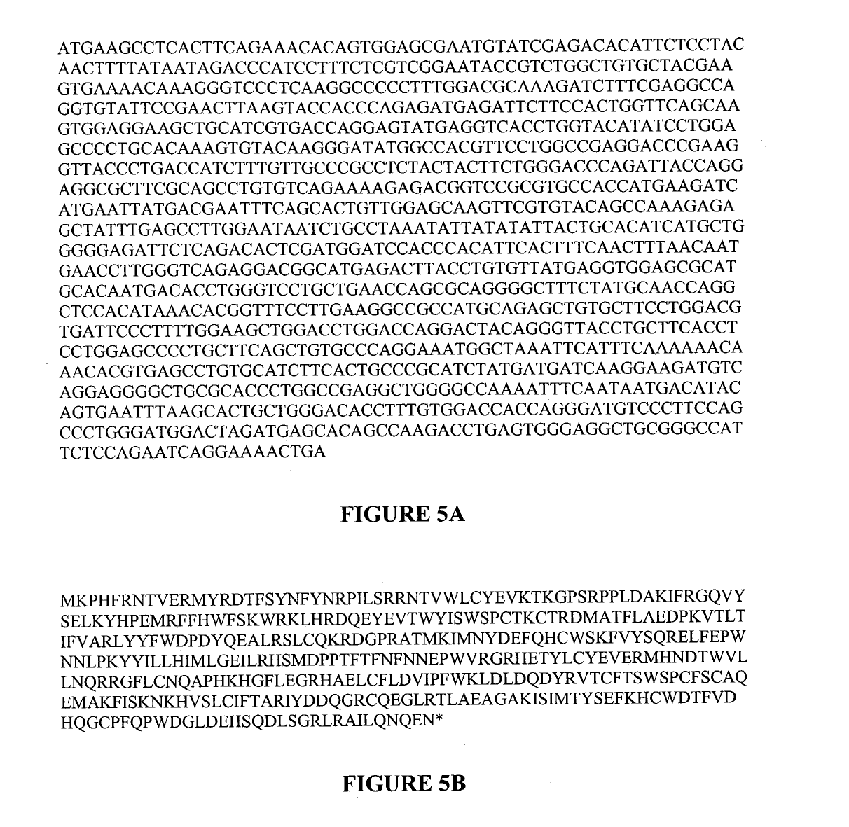

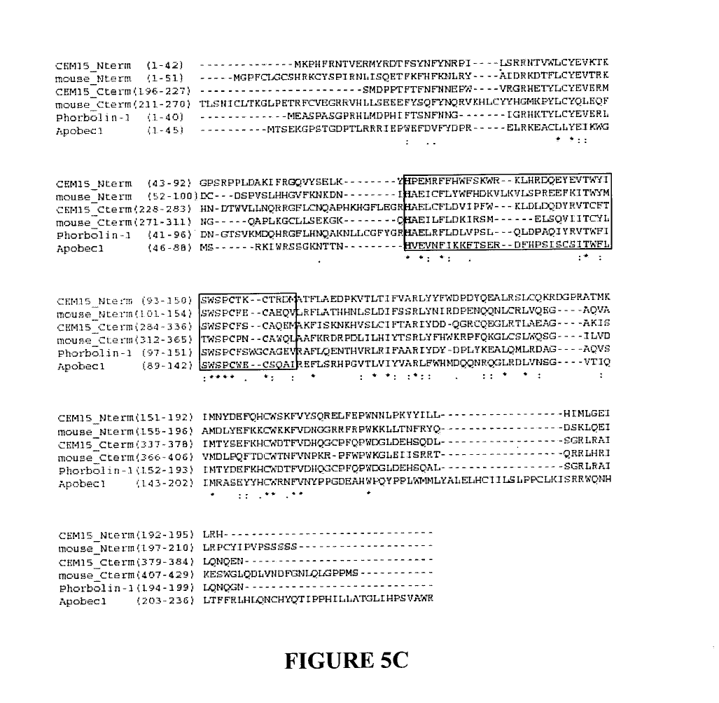

In sum, CEM15 is an efficient and specific inhibitor of HIV-1/Δvif infectivity. Because CEM15 is selectively expressed in NP cells (FIG. 2), and its anti-HIV phenotype is overcome by the levels of Vif expressed during normal HIV-1 replication (FIG. 4), it is evident that the CEM15 protein is the human cell target for Vif function. Later experiments have confirmed the provocative sequence similarity between CEM15 and cytosine deaminases (FIG. 5) by establishing its catalytic role in the deamination of retroviral DNA.

Notably, the inhibitory action of CEM15 during HIV-1 assembly and virion production stands in contrast to the anti-retroviral effects of Fv1 and Ref1, which act in target cells to restrict infection by incoming murine leukemia virus (MLV) particles (Pryciak, et al., J. Virol. 66:5959-5966 (1992); Towers et al., Proc. Natl. Acad. Sci. USA 97:12295-12299 (2000)). Consequently, further testing was conducted, as described in the Examples that follow, to confirm that CEM15 comprises a significant part (or all) of a novel form of innate anti-viral resistance that acts during the late stages of the viral life cycle, and that relieving its suppression by Vif lends a fresh perspective to the area of HIV/AIDS therapeutics.

Thus, in preferred embodiments, the present invention provides at least the following characteristics regarding CEM15 (although these findings are not intended to be limiting):

1) CEM15 is responsible for the massive deamination of deoxycytidine observed in the first cDNA (minus) strand of the retrovirus (detected as G to A transition mutations in the plus strand). dG->dA changes in the HIV plus-strand DNA can be directly explained by modifications of deoxycytidine to deoxyuridine (dC->dU) in the minus-strand of DNA by a DNA deaminase, because U is subsequently read as T by DNA polymerases. Such changes have been wrongly attributed previously as hypermutation by reverse transcriptase, a phenomenon markedly accentuated by low intracellular deoxycytidine triphosphate to deoxythymidine triphosphate (dCTP/dTTP) ratios (Martinez et al., Proc. Natl. Acad. Sci. U.S.A. 91, 11787 (1994)). Therefore the discovery of CEM15 functioning by deamination of retroviral DNA is a giant conceptual leap as the mode of CEM15 innate defense against HIV (also reported by Sheehy et al., 2002), which prior to the findings by the inventors was completely unknown, as was its relationship to RNA editing of viral mRNA (genome) or a host message essential for virus maturation (as predicted incorrectly by many). Attribution of this gene to DNA deamination is unambiguously supported by the present findings, which demonstrate the most central aspects of this mechanism including the DNA deaminase role of CEM15.

2) CEM15 can in fact deaminate deoxycytidine in single-strand DNA, lending further support to preceding point 1).

3) CEM15 is incorporated into MLV virions (as it is into HIV), making this the apparent route for delivery of the producer cell-derived antiviral effect. Thus, CEM15 becomes in effect a molecular time bomb, which is capable of destroying the virus upon entry into a new (target) cell, which is a novel exploitation of the retroviral life-cycle.

4) CEM15-dependent diminution of MLV infectivity (as monitored by fluorescence) can be suppressed by the reintroduction of HIV Vif. This strongly supports the teaching that the proposed mechanism of operation revealed in the present invention is likely to be a general mode of innate immunity, possibly even pre-dating the AID-dependent antibody diversification program.

To our knowledge this is the first demonstration of purposeful mutation of a pathogen by a human protein. It is a most probable contributor to the observed variation in HIV (possibly associated with drug-resistance and immune evasion).

In a preferred embodiment, the present invention further provides a facile MLV-based assay for monitoring this innate immune response, a point that will surely influence those interested in further studying the mechanism and in implementing viral-based gene therapies (as growth of constructs on CEM15-expressing producer cells is likely to confuse the experimental/clinical outcomes).

Notably, subsequent to the findings of the present invention, it was reported that when HIV replicates in cells expressing CEM15, the Vif protein, through mechanisms that remain to be defined, is able to prevent the accumulation of multiple defects in structural, enzymatic, and regulatory viral proteins that would otherwise result in failure at several points in the HIV life cycle (Lecossier et al., Science 300:1112 (May 16, 2003)). In contrast to the present invention, however, the Lecossier et al. report is based on a small database of mutations apparent in Vif-deficient, but not wildtype HIV. Although no direct link is provided, Lecossier et al. speculate that CEM 15 is responsible for the anti-viral phenotype. However, their reported observations are equally consistent with a preferential reverse transcriptase-dependent mis-incorporation of dU during first strand cDNA synthesis. Thus, the DNA deaminase effect of CEM15, provided in the present invention, is neither suggested, nor proven by Lecossier et al.

As previously noted, CEM15 (also APOBEC3G) is a member of the APOBEC family of proteins. Accordingly, although defined by SEQID NO:2 and encoded by gene SEQID NO: 1, various sequences equivalent to CEM15 will be effective that vary from the defined sequences, but are intended to be encompassed by the present invention differs from that of previously known viruses. The present invention, therefore, relates to those compositions, and corresponding DNA and amino acid sequences, which correspond to a large extent to the sequence of CEM15 according to the invention, the degree of deviation being established by the degree of homology. The invention includes a recombinant cell comprising an isolated nucleic acid wherein the nucleic acid shares at least about 50%, preferably at least about 70%, more preferably at least 80% identity with SEQID NO:1. An homology of, for example, more than 80% denotes, therefore, that those sequences are included which have in at least 80 of 100 nucleotides (or in the case of proteins, amino acids) the same nucleotides or amino acids, respectively, while the remainder can be different.

When establishing homology, the two sequences are compared in such a way that the greatest possible number of nucleotides or amino acids corresponding to each other are placed in congruence. However, for the purposes of this invention, the resulting activity of the variant must be equivalent to CEM15. In particular, certain other members of the APOBEC family have proven to have anti-viral effects, and the gene homologues encoding them are encompassed by the present invention. Thus, the present invention includes, without limitation, proteins, polypeptides and oligopeptides derived therefrom which can be used diagnostically or can be employed as vaccines.

The present invention also provides for using analogs of proteins or peptides capable of neutralizing or inhibiting a viral infection. Analogs can differ from naturally occurring proteins or peptides by conservative amino acid sequence differences or by modifications which do not affect sequence, or by both. The invention includes analogs comprising a purified amino acid sequence sharing at least about 50% identity, preferably at least about 70%, more preferably at least about 80% identity with SEQID NO:2, with the proviso that such analogs provide CEM15 activity. In particular, other members of the APOBEC family having anti-viral effects are encompassed by the present invention.

For example, conservative amino acid changes may be made which, although they alter the primary sequence of the protein or peptide, do not normally alter its function. Conservative amino acid substitutions typically include substitutions within the following groups: glycine, alanine; valine, isoleucine, leucine; aspartic acid, glutamic acid; asparagine, glutamine; serine, threonine; lysine, arginine; phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences, which have phosphorylated amino acid residues, e.g., phosphotyrosine, phosphoserine, or phosphothreonine.

Also included are polypeptides, which have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

In addition to substantially full length polypeptides, the present invention provides for enzymatically active fragments of the polypeptides.

As used herein, the term “an isolated preparation” or a “purified preparation” describes a compound, e.g., a gene or nucleic acid molecule, which has been separated from components which naturally accompany it. Typically, a compound is isolated when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method. A compound, e.g., a gene or nucleic acid molecule, is also considered to be isolated when it is essentially free of naturally associated components or when it is separated from the native contaminants which accompany it in its natural state.

The nucleic acid can be duplicated using a host-vector system and traditional cloning techniques with appropriate replication vectors. A “host-vector system” refers to host cells, which have been transfected with appropriate vectors using recombinant DNA techniques. The vectors and methods disclosed herein are suitable for use in host cells over a wide range of eucaryotic organisms. This invention also encompasses cells transformed with the novel replication and expression vectors described herein.

The selected gene, made and isolated using the above methods, can be directly inserted into an expression vector, such as described herein, and inserted into a suitable animal or mammalian cell, such as a guinea pig cell, a rabbit cell, a simian cell, a mouse, a rat or a human cell.

In the practice of one embodiment of this invention, the modulating gene, such as the purified CEM15 nucleic acid molecule is introduced into the cell and expressed, thereby inhibiting viral infectivity. A variety of different gene transfer approaches are available to deliver the gene or gene fragment encoding the modulating nucleic acid into a target cell, cells or tissues. Among these are several non-viral vectors, including DNA/liposome complexes, and targeted viral protein DNA complexes. To enhance delivery to a cell, the nucleic acid or proteins of this invention can be conjugated to antibodies, or binding fragments thereof, which bind cell surface antigens. Liposomes that also comprise a targeting antibody or fragment thereof can be used in the methods of this invention. This invention also provides the targeting complexes for use in the methods disclosed herein.

Suitable cells or “target cells” for the practice of the present invention include, but are not limited to, cells, cell populations and tissues affected by changes affected by CEM15.

Non-viral techniques may include, but are not limited to colloidal dispersion, asialorosonucoid-polylysine conjugation, or, less preferably, microinjection under surgical conditions

The nucleic acid molecule encoding the modulator composition also can be incorporated into a “heterologous DNA” or “expression vector” for the practice of this invention. The term “heterologous DNA” is intended to encompass a DNA polymer such as viral vector DNA, plasmid vector DNA or cosmid vector DNA. Prior to insertion into the vector, it is in the form of a separate fragment, or as a component of a larger DNA construct, which has been derived from DNA isolated at least once in “substantially pure form,” i.e., free of contaminating endogenous materials and in a quantity or concentration enabling identification, manipulation, and recovery of the segment and its component nucleotide sequences by standard biochemical methods, for example, using a cloning vector.

As used herein, “recombinant” is intended to mean that a particular DNA sequence is the product of various combination of cloning, restriction, and ligation steps resulting in a construct having a sequence distinguishable from homologous sequences found in natural systems. Recombinant sequences can be assembled from cloned fragments and short oligonucleotides linkers, or from a series of oligonucleotides.

As noted above, one means to introduce the nucleic acid into the cell of interest is by the use of a recombinant expression vector. “Recombinant expression vector” is intended to include vectors, which are capable of expressing DNA sequences contained therein, where such sequences are operatively linked to other sequences capable of effecting their expression. It is implied, although not always explicitly stated, that these expression vectors must be replicable in the host organisms either as episomes or as an integral part of the chromosomal DNA.

Accordingly, “expression vector” is given a functional definition, and any DNA sequence which is capable of effecting expression of a specified DNA sequence disposed therein is included in this term as it is applied to the specified sequence. Suitable expression vectors include viral vectors, including adenoviruses, adeno-associated viruses, retroviruses, cosmids and others. Adenoviral vectors are a particularly effective means for introducing genes into tissues in vivo because of their high level of expression and efficient transformation of cells both in vitro and in vivo.

Expression levels of the gene or nucleotide sequence inside a target cell are capable of providing gene expression for a duration and in an amount such that the nucleotide product therein is capable of providing a therapeutically effective amount of gene product or in such an amount as to provide a functional biological effect on the target cell.

By “gene delivery” is meant transportation of a composition or formulation into contact with a target cell so that the composition or formulation is capable of being taken up by means of a cytotic process (i.e., pinocytosis, endocytosis, phagocytosis, and the like) into the interior or cytoplasmic side of the outermost cell membrane of the target cell where it will subsequently be transported into the nucleus of the cell in such functional condition that it is capable of achieving gene expression.

By “gene expression” is meant the process, after delivery into a target cell, by which a nucleotide sequence undergoes successful transcription and translation such that detectable levels of the delivered nucleotide sequence are expressed in an amount and over a time period that a functional biological effect is achieved. “Gene therapy” encompasses the terms ‘gene delivery’ and ‘gene expression.’ Moreover, treatment by any gene therapy approach may be combined with other, more traditional therapies.

Replication-incompetent retroviral vectors also can be used with this invention. As used herein, the term “retroviral” includes, but is not limited to, a vector or delivery vehicle, many of which are known in the art, having the ability, via an RNA to DNA lifecycle, to selectively target and introduce the coding sequence into dividing or non-dividing cells. As used herein, the terms “replication-incompetent” is defined as the inability to produce viral proteins, precluding spread of the vector in the infected host cell. As would be understood by those of skill in the art, the nucleic acid introduced with a retroviral vector would be a DNA, but would be packaged in the context of the virus as ribonucleic acid (RNA). The methodology further includes using replication-incompetent retroviruses for retroviral-mediated gene.

The invention further includes a vector comprising a gene encoding a composition for modulating viral infectivity as compared with a selected standard of activity or for cells or tissues grown without the modulator or disclosed method effecting such a change. DNA molecules composed of a protein gene or a portion thereof, can be operably linked into an expression vector and introduced into a host cell to enable the expression of these proteins by that cell. Alternatively, a protein may be cloned in viral hosts by introducing the “hybrid” gene operably linked to a promoter into the viral genome. The protein may then be expressed by replicating such a recombinant virus in a susceptible host. A DNA sequence encoding a protein molecule may be recombined with vector DNA in accordance with conventional techniques. When expressing the protein molecule in a virus, the hybrid gene may be introduced into the viral genome by techniques well known in the art. Thus, the present invention encompasses the expression of the desired proteins in either prokaryotic or eukaryotic cells, or viruses that replicate in prokaryotic or eukaryotic cells.

Preferably, the proteins of the present invention are cloned and expressed in a virus. Viral hosts for expression of the proteins of the present invention include viral particles that replicate in prokaryotic host, or viral particles that infect (transfect) and replicate in eukaryotic hosts. Procedures for generating a vector for delivering the isolated nucleic acid or a fragment thereof, are well known, and are described for example in Sambrook et al. Suitable vectors include, but are not limited to, disarmed adenovirus, bovine papilloma virus, simian virus and the like.

Once the vector or DNA sequence containing the constructs has been prepared for expression, the DNA constructs may be introduced or transformed into an appropriate host. Various techniques may be employed, such as protoplast fusion, calcium phosphate precipitation, electroporation, or other conventional techniques, see e.g., the examples which follow. As is well known, viral sequences containing the “hybrid” protein gene may be directly transformed into a susceptible host or first packaged into a viral particle and then introduced into a susceptible host by infection. After the cells have been transformed with the recombinant DNA (or RNA) molecule, or the virus or its genetic sequence is introduced into a susceptible host, the cells are grown in media and screened for appropriate activities. Expression of the sequence results in the production of protein(s) of the present invention.

Also using the isolated sequence as a basis, immunodominant epitopes (peptides) can be designed and synthesized. Since the nucleic acid sequence of the virus is known, the person skilled in the art can derive the amino acid sequence from this known sequence. The present invention also relates, therefore, to antigens, i.e., proteins, oligopeptides or polypeptides, which can be prepared with the aid of the information disclosed herein. These peptides can be prepared not only with the aid of recombinant technology, but also using synthetic methods. A suitable preparation route is solid-phase synthesis of the Merrifield type. Further description of this technique, and of other processes known to the state of the art, can be found in the literature, e.g., M. Bodansky et al., Peptide Synthesis, John Wiley & Sons, 2nd Edition 1976.

The expression of the desired protein in eukaryotic hosts requires the use of eukaryotic regulatory regions. Such regions will, in general, include a promoter region sufficient to direct the initiation of RNA synthesis. As is widely known, translation of eukaryotic mRNA is initiated at the codon, which encodes the first methionine. For this reason, it is preferable to ensure that the linkage between a eukaryotic promoter and a DNA sequence which encodes the desired protein does not contain any intervening codons which are capable of encoding a methionine (i.e., AUG).

The desired protein encoding sequence and an operably linked promoter may be introduced into a recipient prokaryotic or eukaryotic cell either as a non-replicating DNA (or RNA) molecule, which may either be a linear molecule or, more preferably, a closed covalent circular molecule. Since such molecules are incapable of autonomous replication, the expression of the desired protein may occur through the transient expression of the introduced sequence. Alternatively, permanent expression may occur through the integration of the introduced sequence into the host chromosome. For expression of the desired protein in a virus, the hybrid gene operably linked to a promoter is typically integrated into the viral genome, be it RNA or DNA. Cloning into viruses is well known and thus, one of skill in the art will know numerous techniques to accomplish such cloning.

Cells which have stably integrated the introduced DNA into their chromosomes can be selected by also introducing one or more reporter genes or markers which allow for selection of host cells which contain the expression vector. The reporter gene or marker may complement an auxotrophy in the host, or provide biocide resistance, e.g., antibiotics. The selectable marker gene can either be directly linked to the DNA gene sequences to be expressed, or introduced into the same cell by co-transfection, e.g., luciferase in the Examples that follow.

Additional elements may also be needed for optimal synthesis of mRNA. These elements may include splice signals, as well as transcription promoters, enhancers, and termination signals. The cDNA expression vectors incorporating such elements include those described by Okayama, H., Mol. Cell. Biol. 3:280 (1983), and others.

The method can also be practiced ex vivo using a modification of recognized methods. Generally, a sample of cells, such as T cells can be removed from a subject or animal using methods well known to those of skill in the art. An effective amount of the selected nucleic acid or polypeptide is added to the cells, and the cells are cultured under conditions that favor internalization of the homologous or heterologous component by the cells. The transformed cells are then returned or reintroduced to the same subject or animal (autologous) or one of the same species (allogeneic) in an effective amount and in combination with appropriate pharmaceutical compositions and carriers. Alternatively, they may be used for research purposes.

By “patient” or “subject” is meant any vertebrate or animal, preferably a mammal, most preferably a human, with a viral infection, particularly with a retroviral infection, such as HIV-1, of at least some portion of a population of cells, or with a susceptibility to, or genetic predisposition to such infection in such selected population of cells. Thus, included within the present invention are animal, bird, reptile or veterinary patients or subjects, the intended meaning of which is self-evident. Despite notable differences in anatomy between the certain physical features of a primate and those of a rodent or bird, at the cellular level, cell death among other animals closely resembles that in the primate, as shown by studies made on rats, chicks and young monkeys. All are encompassed by the methods of the present invention.

The invention further defines methods for modulating viral infectivity in a patient or host, or in the cells or tissues of a patient or host. In a preferred embodiment the method inhibits or suppresses viral infectivity, although there may be occasions in which the methods described herein may be applied in selected embodiments to enhance or initiate viral infectivity. By “inhibition” or “suppression” is meant a statistically significant reduction in viral infectivity as a result of the administration of or expression of CEM15, as compared with a selected standard of activity or for cells or tissues grown without the inhibitor or disclosed method of inhibition. Preferably, the inhibitor changes viral infectivity by at least 20%, more preferably by at least 50%, even more preferably by 80% or greater, and also preferably, in a dose-dependent manner. Once inhibitors satisfying these requirements are identified, the utilization of assay procedures to identify the manner in which the viral infectivity is inhibited are particularly useful.

In a preferred embodiment the method prevents viral infectivity. By “prevent” or “prevention” is meant a statistically significant reduction in the onset of viral infectivity as a result of the administration of or expression of CEM15 or related agents, as compared with a selected standard of activity or for cells or tissues grown without the agent or disclosed method of prevention. As used hereing, the term prevent or prevention is not intended to be an absolute term, necessarily meaning that in all cases onset of viral infectivity is 100% blocked, although the prevention may reach effectively 100%. Preferably, the preventor blocks or protects against viral infectivity by at least 20%, more preferably by at least 50%, even more preferably by 80% or greater, and also preferably, in a dose-dependent manner. Once preventors satisfying these requirements are identified, the utilization of assay procedures to identify the manner in which the viral infectivity is prevented are particularly useful.

In yet another embodiment, it may be useful particularly in a research environment to apply methods in which viral infectivity is initiated or enhanced. By “enhance” or “initiate” viral infectivity is meant a statistically significant increase in viral infectivity as a result of the administration of or expression of CEM15 or related agent, as compared with a selected standard of activity or for cells or tissues grown without the agent or disclosed methods. Preferably, the enhancer or initiator increases viral infectivity by at least 20%, more preferably by at least 50%, even more preferably by 80% or greater, and also preferably, in a dose-dependent manner. Once enhancers or initiators satisfying these requirements are identified, the utilization of assay procedures to identify the manner in which the viral infectivity is increased are particularly useful.

To determine whether CEM15 of the invention could inhibit or prevent HIV infection, cytopathic effect-(CPE-) based infection experiments were performed as described below. The results of these studies indicate that CEM15 can both inhibit an existing infection and protect against such infection. Even if the preparation of vaccines against immunodeficiency diseases is proving to be extremely difficult, this virus, too, or parts thereof, i.e., immunodominant epitopes and inducers of cellular immunity, or antigens prepared by genetic manipulation, can still be used for developing and preparing vaccines.

A further option for using the virus treated in accordance with the invention in test systems is its use in Western blots, screening assays and high through-put screening for determining therapeutic formulations effective against viral or retroviral infection, particularly for inhibiting HIV-1. In this method, a test cell is contacted with CEM15 or placed in the presence of a CEM15 expressing cell of the invention such that virus present in the test cell at the time of contact, or after such contact is unable to replicate. In embodied diagnostic tests, a serum sample from the person to be investigated is brought into contact with the protein chains of one or more proteins or glycoproteins (which can be expressed in eukaryotic cell lines), or parts thereof, which originate from CEM15. Test processes which are preferred include immunofluorescence or immunoenzymatic test processes (e.g. ELISA or immunoblot).

In yet another embodiment, there are provided methods for treating a subject susceptible to virus, particularly retrovirus such as HIV-1, infection. The CEM15 or “CEM15 expressing cell' (or “CEM15 in a virus-producer cell”), preferably as “Vif-resistant forms of CEM15 in a CEM15 expressing cell,” as described herein are administered to the mammal in the form of therapeutic pharmaceutical formulations that are effective for treating virus infection. The pharmaceutical formulation may be administered in conjunction with other therapeutic agents, e.g., AZT and/or various protease inhibitors, to treat AIDS.

Administration of pharmaceutical compositions in accordance with invention or to practice the method of the present invention can be carried out in a variety of conventional ways, such as by oral ingestion, enteral, colorectal, or transdermal administration, inhalation, sublingual administration, or cutaneous, subcutaneous, intramuscular, intraocular, intraperitoneal, or intravenous injection, or any other route of administration known in the art for administrating therapeutic agents.

The therapeutic pharmaceutical formulation containing at least one virus-producer cell containing CEM15 or a CEM15 expressing cell, according to the invention, includes a “pharmaceutically or physiologically acceptable carrier” includes any and all solvents, dispersion media, coatings, antibacterial and antifungal agents, isotonic and absorption delaying agents and the like, which are congruent with the mode of administration. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions. Examples include an inert diluent or a biologically-acceptable edible carrier. Suitable formulations that include pharmaceutically acceptable excipients for introducing compounds to the bloodstream by intravenous injection and other than injection routes can be found in Remington's Pharmaceutical Sciences (18th ed.) (Genarro, ed. (1990) Mack Publishing Co., Easton, Pa.).

The pharmaceutical forms suitable for injectable use include sterile aqueous solutions or dispersions and sterile powders for the extemporaneous preparation of sterile injectable solutions or dispersions. In all cases the form must be sterile. It must be stable under the conditions of manufacture and storage and may be preserved against the contaminating action of microorganisms, such as bacterial and fungi. The carrier can be a solvent or dispersion medium. The prevention of the action of microorganisms can be brought about by various antibacterial and antifungal agents. Prolonged absorption of the injectable therapeutic agents can be brought about by the use of the compositions of agents delaying absorption. Sterile injectable solutions are prepared by incorporating the CEM15 expressing cell in the required amount in the appropriate solvent, followed by filtered sterilization.

When a therapeutically effective amount of composition of the invention is administered by injection, the CEM15 expressing cell(s) will preferably be in the form of a pyrogen-free, parenterally-acceptable, aqueous solution. The preparation of such parenterally-acceptable solutions, having due regard to pH, isotonicity, stability, and the like, is within the skill in the art. A preferred pharmaceutical composition for injection should contain, in addition to the CEM15 expressing cell(s), an isotonic vehicle such as sodium chloride injection, Ringer'injection, dextrose injection, dextrose and sodium chloride injection, lactated Ringer's injection, or other vehicle as known in the art. The pharmaceutical composition of the present invention may also contain stabilizers, preservatives, buffers, antioxidants, or other additives known to those of skill in the art. The pharmaceutical formulation can be administered in bolus, continuous, or intermittent dosages, or in a combination of continuous and intermittent dosages, as determined by the physician and the degree and/or stage of illness of the patient.

The duration of therapy using the pharmaceutical composition of the present invention will vary, depending on the unique characteristics of the CEM15 expressing cell(s) and the particular therapeutic effect to be achieved, the limitations inherent in the art of preparing such a therapeutic formulation for the treatment of humans, the severity of the disease being treated and the condition and potential idiosyncratic response of each individual patient. Ultimately the attending physician will decide on the appropriate duration of intravenous therapy using the pharmaceutical composition of the present invention.

Alternatively, the CEM15 expressing cell of the invention and other ingredients may be enclosed in a hard or soft shell gelatin capsule, compressed into tablets, or incorporated directly into the individual's diet. The CEM15 expressing cell(s) may be incorporated with excipients and used in the form of ingestible tablets, buccal tablets, troches, capsules, elixirs, suspensions, syrups, wafers, and the like. When the CEM15 expressing cell(s) are administered orally, it may be mixed with other food forms and pharmaceutically acceptable flavor enhancers. When the CEM15 expressing cell(s) is administered enterally, they may be introduced in a solid, semi-solid, suspension, or emulsion form and may be compounded with any number of well-known, pharmaceutically acceptable additives.

Sustained release oral delivery systems and/or enteric coatings for orally administered dosage forms are also contemplated such as those described in U.S. Pat. Nos. 4,704,295, 4,556,552, 4,309,404, and 4,309,406.

As used herein, the term “therapeutically effective amount” means the total amount of each active component of the pharmaceutical formulation or method that is sufficient to show a meaningful subject or patient benefit, i.e., a reduction in viral infectivity or the expression of proteins which activate or enhance an innate anti-viral mechanism. This is also referred to as a “sufficient amount.” When applied to an individual active ingredient, administered alone, the term refers to that ingredient alone. When applied to a combination, the term refers to combined amounts of the active ingredients that result in the therapeutic effect, whether administered in combination, serially or simultaneously.

A “therapeutically effective manner” refers to a route, duration, and frequency of administration of the pharmaceutical formulation, which ultimately results in meaningful patient benefit, as described above. In some embodiments of the invention, the pharmaceutical formulation is administered via injection, sublingually, colorectally, intradermally, orally, enterally or in bolus, continuous, intermittent, or continuous, followed by intermittent regimens.

The therapeutically effective amount of CEM15 expressing cell(s) administered in the method of the invention will depend upon the nature and severity of the condition being treated, and on the nature of prior treatments which the patent has undergone. Ultimately, the attending physician will decide the amount of synthetic CEM15 or CEM15 expressing cell(s) with which to treat each individual patient. Initially, the attending physician may administer low doses of CEM15 or the CEM15 expressing cell(s) and observe the patient's response. Larger doses of CEM15 or the CEM15 expressing cell(s) may be administered until the optimal therapeutic effect is obtained for the patient, and at that point the dosage is not increased further. It is contemplated that the dosages of the pharmaceutical compositions administered in the method of the present invention should contain about 0.1 to 100.0 mg/kg body weight per day, preferably 0.1 to 75.0 mg/kg body weight per day, more preferably, 1.0 to 50.0 mg/kg body weight per day, even more preferably, 1 to 25 mg/kg body weight per day, and even more preferably, 1 to 10 or 1 to 5.0 mg/kg body weight per day.

However, for localized administration, much lower concentrations than this may be effective, and much higher concentrations may be tolerated. It may be desirable to administer simultaneously or sequentially a therapeutically effective amount of one or more of the therapeutic compositions of the invention when individual as a single treatment episode.

It will be appreciated that the unit content of active ingredient or ingredients contained in an individual dose of each dosage form need not in itself constitute an effective amount since the necessary effective amount can be reached by administration of a plurality of dosage units (such as suppositories, gels, or creams, or combinations thereof). In fact, multi-dosing (once a day) has been shown to significantly increase the plasma and tissue concentrations of MBO's (data not shown).

In addition, if systemically administered CEM15 formulations will cross the placenta in pregnant females, the formulations would be available in the blood of embryos in utero. Thus, it is contemplated that formulations of the invention may be used in a method of treating the fetuses and human mothers harboring HIV.

The following examples illustrate the preferred modes of making and practicing the present invention, but are not meant to limit the scope of the invention since alternative methods may be utilized to obtain similar results.

### EXAMPLES

### Example 1

### Anti-viral Function of CEM15

As noted above, cell fusion experiments have established that the NP phenotype is dominant over the P phenotype in that Δvif virions produced from NP−P heterokaryons are non-infectious. Consequently, a model is supported in which NP cells selectively express an anti-viral activity that is suppressed by Vif, and predicts that NP cells express genes whose ectopic expression in P cells should phenotypically convert them to NP cells. To identify candidate genes of this type, a PCR-based cDNA subtraction strategy in which the NP sample served as the “tester” and the P sample was used as the “driver.” (The PCR system has proved to have a multiplicity of uses in genetic manipulation, and is well understood in the art. The necessary components implementing the process are commercially available.) To enrich for clones specific to the NP phenotype, a pair of genetically-related cell lines were identified that exhibit distinct abilities to support Δvif virus replication.

Throughout the following experiments, all lymphoid and non-lymphoid cell lines were maintained under standard conditions. Peripheral blood mononuclear cells (PBMCs) were obtained by venipuncture, stimulated with 5 μg/ml phytohemagglutinin-L and maintained in medium supplemented with interleukin-2. Monolayer cultures of 293T cells were transiently transfected using calcium phosphate, and CEM-SS cells were transduced using a murine retroviral transduction system (Fouchier, et al., EMBO J. 16:4531-4539 (1997)) followed by maintenance in medium containing 1 mg/ml G418.

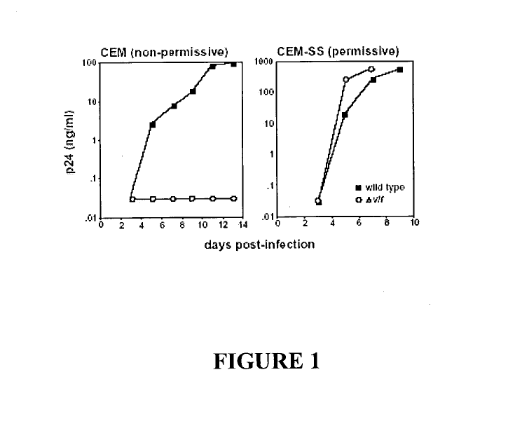

CEM-SS is a subclonal isolation of the CEM parental T cell line (Foley et al., Cancer Research 25: 1254-1261 (1965); Nara et al., Nature 332: 469-470 (1988)) and the genetic relatedness of these two lines was confirmed by sequencing the V(D)J DNA rearrangement junctions at the T cell receptor γ locus (data not shown). However, when the CEM (NP) and CEM-SS (P) T cells were challenged with normalized stocks of wild type or Δvif X4-tropic viruses, the NP phenotype of the CEM line was confirmed by its inability to support a spreading infection of HIV-1/Δvif. Stocks of wild type and Δvif viruses were prepared by transfection of 293T cells, quantitated by ELISA for p24^{Gag} content and stored at −80° C. Spreading infections were initiated with stocks normalized according to p24^{Gag} levels, the cells passaged, and replication monitored over time as the accumulation of p24^{Gag} in the culture supernatants. Single-cycle infectivities were determined by challenging the C8166-CCR5/HIV-CAT indicator cell line (5×10⁵ cells) with viruses corresponding to 5 ng p24^{Gag}, and measuring the induced expression of chloramphenicol acetyl transferase (CAT) in cell lysates after ˜28 hours (Simon et al., J. Virol. 70, 5297-5305 (1996)). By comparison, the P phenotype of CEM-SS cells was verified by the indistinguishable replication profiles of wild type and Δvif viruses (FIG. 1).

Preparations of polyadenylated RNA (Qiagen Oligotex mRNA Midi Kit, Valencia, Calif.) derived from wild type HIV-1 infected (˜50% antigen positive) CEM or CEM-SS cells were reverse transcribed and subjected to subtraction using the PCR-Select system (Clontech, Palo Alto, Calif.) with the CEM sample serving as the “tester.” Ensuing subcloned cDNA fragments were sequenced and used as probes in Northern blotting screens of RNAs isolated from a panel of NP and P cells (data not shown). A cDNA encoding the predicted 384 amino acid ORF of an novel gene, named CEM15 by the inventors, was derived using RT-PCR and inserted into the EcoRI site of pcDNA3.1 (Invitrogen, Carlsbad, Calif.). Three copies of the sequence encoding the influenza virus HA epitope-tag (Field et al., Mol. Cell Biol. 8:2159-2165 (1988)) were included at the 3′-end of CEM15 to create pCEM15:HA.

The wild type and Δvif X4 provirus expression vectors, pIIIB and pIIIB/Δvif, have been described (Simon et al., J. Virol. 70, 5297-5305 (1996)), as has the wild type R5 proviral vector, pYU-2 (Fouchier et al., 1997). A Δvif derivative of pYU-2 was generated by removal of nucleotides 5126 to 5138. The MSCV-based retroviral vector NG/neo was derived from MIGR1 (Pear et al., Blood 92:3780-3792 (1998)) by replacement of the GFP gene with the neomycin phosphotransferase gene, and the CEM15:HA cassette was then inserted as an XhoI-EcoRI fragment to create NG/C15. The luciferase expression vector, pLUC, was constructed by insertion of the luciferase gene from pBI-L (Clontech) into pcDNA3.

Individual subtracted cDNAs were used as probes in Northern blot analyses of RNAs extracted from a panel of NP and P cells (FIG. 2). To accomplish this, total RNAs were isolated and extracted from infected or uninfected cells using standard procedures, ˜5 μg of which was then electrophoretically resolved on MOPS-containing agarose gels and transferred to GeneScreen (PerkinElmer Life Sciences Inc., Boston, Mass.). The filters were hybridized to random primed ³²P-labeled DNA probes corresponding to subtracted cDNAs, and visualized by autoradiography. Following autoradiography, the filter was stripped and rehybridized with a β-actin specific probe to establish equivalent loading of the gel.

The cDNA corresponding to the CEM15 gene yielded a particularly striking result in which a single transcript of ˜1.5 kb was readily detected in all NP cells tested (FIG. 2, lanes 1 to 7), including peripheral blood mononuclear cells. In contrast, however, minimal expression (lanes 10 to 12) or no expression (lanes 8, 9, 13 and 14) was observed in a variety of lymphoid and non-lymphoid P cell lines. Of note, expression levels of CEM15 mRNA were not influenced significantly by HIV-1 infection (lanes 2, 4, 6, 9 and 11).

Because the expression profile for CEM15 corresponds with that which is expected for a gene important for the NP phenotype, the effects of CEM15 expression on HIV-1 (HIV-1/Δvif) particle production and infectivity was examined using transiently transfected 293T cells (a P cell line). 60-mm diameter 293T monolayers were transfected with cocktails comprising 3 μg provirus (wild type or Δvif), 12 μg pcDNA3.1-based vector (titration of pCEM15:HA, with the empty parental vector making up the balance), and 100 ng pLUC. Varying doses (0, 0.5, 1 or 3 μg) of pCEM15:HA (a vector that encodes an epitope-tagged version of the predicted 385 amino acid open reading frame of CEM15) were co-transfected with either wild type or Δvif versions of an X4 provirus expression vector and an internal control luciferase expressing plasmid.

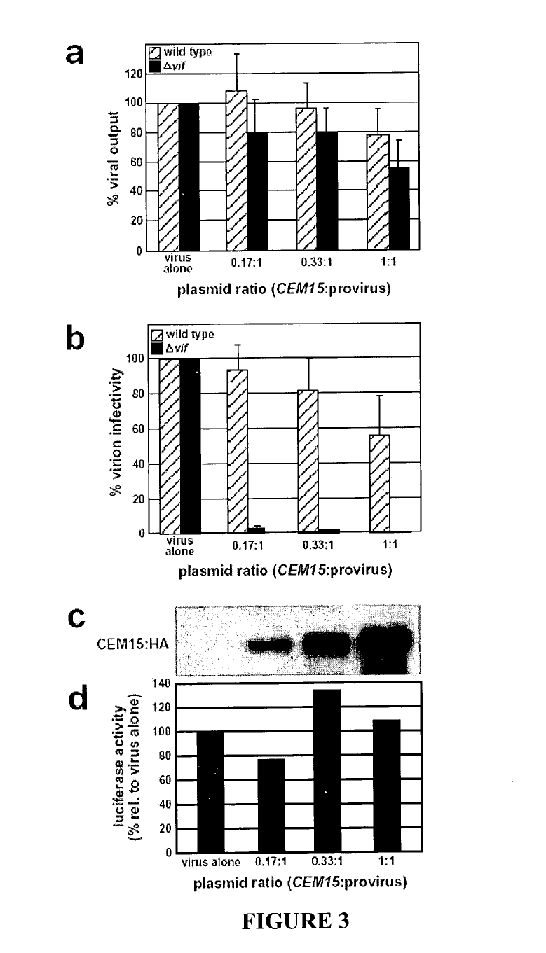

Virus-containing culture supernatants were harvested ˜24 hours post-transfection, and virus production was quantified by ELISA in terms of ng/ml P24^{Gag} levels in the supernatants. The mean values and standard deviations for eight independent experiments are shown in FIG. 3A. Identical results were obtained using a CEM 15 expression vector that lacked an epitope tag (data not shown). The levels of virus output were found to be relatively unaffected for either virus by the presence of CEM15 (FIG. 3A).

These same virus-containing supernatants were then normalized according to p24^{Gag}content, and the viruses corresponding to 5 ng p24^{Gag} were used in single round challenges of C8166-CCR5/HIV-CAT cells (an indicator cell line in which productive HIV-1 infection is registered as the induction of CAT expression). Productive infection was measured as the induction of CAT activity (FIG. 3B). The values are presented in FIG. 3B, as % infectivity relative to virus-alone samples (i.e., no pCEM15:HA used), and the data again represent eight experiments.

Whole cell lysates from one set of Δvif transfections were also examined by immunoblotting to confirm the titration curve for CEM15:HA expression (FIG. 3C), and for luciferase expression to establish that CEM15 does not affect cell physiology or gene expression in a pleiotropic manner (FIG. 3D). CEM15:HA or the hnRNP C½ proteins were detected in whole cell lysates of transfected or transduced cultures using the 16B12 (Covance, Inc., Princeton, N.J.) or 4F4 (Simon et al., J. Virol. 70, 5297-5305 (1996)) monoclonal antibodies for primary hybridization and enhanced chemiluminescence. In some cases, luciferase activity was measured in the same lysates using the Luciferase Assay System (Promega Corp., Madison, Wis.).

The luciferase expression of the cell lysates used for CEM15:HA expression were evaluated, and the values are displayed in FIGS. 3Cand 3D as % activity relative to the virus-alone sample. The titration of protein expression was confirmed for one series of Δvif transfections by immunoblot analysis of whole cell. In contrast to the wild type virus which was largely unaffected by CEM15 expression, the Δvif virus displayed minimal levels of infection at all doses of CEM15 tested. For example, ˜97% reduction in infectivity resulted from a six-fold (6X) excess of Δvif provirus over pCEM15:HA, relative to provirus-alone.

Finally, analogous transfection experiments were also carried out using a matched pair of wild type and Δvif R5 proviral clones. The effects of CEM15 on virus infectivity were essentially the same as for the X4 viruses (data not shown), thus demonstrating that the inhibition of Δvif infection by CEM15 is not strain specific. Taken together, these data show that CEM15 bears all the hallmarks expected for a gene that establishes the NP phenotype. In other words, its expression in P cells inhibits Δvif virus production qualitatively (inhibition of infectivity), but not quantitatively.

### Example 2

### Alternative Experiments to Verify Anti-viral Function of CEM15.

To verify the anti-viral function of CEM15 in an alternative experimental configuration, its effect on HIV-1 replication in T cells was determined. CEM-SS cells were stably transduced with the CEM15:HA-encoding retroviral vector NG/C 15, or with the parental vector NG/neo(negative control). The transduced whole cell lysates of the CEM-SS cells were subjected to immunoblotting using antibodies specific for hnRNP C½ (loading control) or the HA epitope tag. Protein expression in the bulk cultures is shown in FIG. 4A. Cultures of both the control and CEM15 expressing cell lines were then challenged either with wild type or Δvif HIV-1 inocula corresponding to 1 ng p24^{Gag} and monitored as for FIG. 1, i.e., spreading infection was monitored as the time dependent accumulation of p24^{Gag} in the culture supernatants (FIG. 4B). As noted above for parental CEM-SS cells in FIG. 1, the neo-expressing control cells supported efficient replication by both viruses, and stable expression of CEM15 in CEM-SS cells selectively inhibited HIV-1/Δvif replication.

By comparison, however, the CEM15 expressing cells allowed robust replication by the wild type virus, but only very low levels of Δvif virus growth (in this experiment, ˜300-fold reduction in output relative to wild type virus after ten days). Although it appears that a lack of adequate CEM15 expression in a subset of the cells accounts for the residual levels of HIV-1/Δvif replication in this culture, it remains possible that other genes my contribute to the complete NP phenotype (Hassaine et al., J. Biol. Chem. 276:16885-16893 (2001)). Nevertheless, these data further demonstrate that ectopic expression of CEM15 is highly effective at converting cells from the P phenotype to the NP phenotype.

An amino acid database search using CEM15 identified a novel gene expressed in hematopoietic cells isolated from myelodysplastic syndrome (MDS) patients (AF 182420, H. sapiens phorbolin-like protein MDS019). However; no further description was available of the gene encoding the MDS019 phorbolin-like protein, or its function, at the time of initial description (Sheehy et al., 2002). An analysis of the CEM15 protein sequence (predicted relative molecular mass, 46,405 Da) revealed a murine orthologue, but no significant matches in S. cerevisiae, D. melanogaster, or C. elegans.

Further analysis (ClustalW alignment) showed substantial similarities among the amino- and carboxyl-terminal domains of human CEM15 (AAG14956), its mouse orthologue (AAH03314), human phorbolin-1 (AAA03706) and human apobec-1 (NP_{—}001635) (the cytidine deaminase that is the catalytic subunit of the mammalian apolipoprotein B mRNA editing enzyme) proteins. Residue numbers for each line of sequence are provided in FIG. 5. Identical, conserved or semi-conserved residues were found, and as demonstrated that the amino- and carboxy-terminal portions of both human and murine CEM15 possess significant amino acid identity to each other, to apobec-1 and to the novel PMA-induced protein phorbolin-1 (FIG. 5) (Madsen et al., J. Invest. Dermatol. 113:162-169 (1999); Teng et al., Science 260:1816-1819 (1993)). For instance, the carboxy-terminal region of human CEM15 exhibits ˜50% amino acid identity to apobec-1 and ˜70% amino acid identity to phorbolin-1.

Particularly intriguing is the delineation of a zinc-coordinating motif in each of these protein domains (FIG. 5, boxed regions indicating the Prosite cytosine nucleoside/nucleotide deaminase zinc-binding motif PS00903, with putative zinc binding histidine and cysteine residues shown in bold). This zinc-binding domain has previously been identified in cytosine nucleoside/nucleotide deaminases present in organisms ranging from bacteriophage to humans (Bhattacharya et al., Trends Biochem. Sci. 19:105-106 (1994)), and has been shown to be critical for the catalytic activity of these enzymes (MacGinnitie et al., J. Biol. Chem. 270:14768-14775 (1995); Smith et al., Biochemistry 33:6468-6474 (1994)). In sum, CEM15 is an efficient and specific inhibitor of HIV-1/Δvif infectivity.

### Example 3

### Expression of CEM15 in Producer Cell Results in Substantial Inhibition of vif-deficient HIV and MLV Infections

To confirm that human CEM15, a potent inhibitor of vif-deficient HIV (HIV/Δvif) (Sheehy et al., Nature 418:646-650 (2002)), also impedes infection by MLV, stocks of recombinant MLV encoding yellow fluorescent protein (YFP) were produced in 293T cells (a line not expressing CEM15) (Sheehy et al., 2002)) in the presence or absence of transfected human CEM15 (Bock et al., J. Virol. 74:7422-7430 (2000)).

The 293T cell derivatives stably expressing CEM15 were selected using 250 μg of Zeocin and expression was confirmed by Northern blot analysis, and the cell stocks were quantified according to relative levels of reverse transcriptase (RT) activity in their supernatants (Cavidi Tech, Uppsala, Sweden). Approximately one-sixth confluent monolayers of Mus dunni (murine) fibroblasts or human 293T cells were challenged with varving doses of YFP- or GFP-encoding MLV stocks (viral inocula measured by RT activity) produced by co-transfection with a control vector, or with 1 μg or 3 μg of pCEM15:HA vector (as described by Sheehy et al., 2002, and Bock et al., J. Virol. 74:7422-7430 (2000)). These were maintained for ˜48 hours, and then productive infection in terms of the proportion of YFP-positive cells was monitored by flow cytometry (Becton Dickinson Immunocytometry Systems, San Jose, Calif.) using mock-infected cells as negative controls (Id).

As seen in FIG. 6A, the presence of CEM15 had a dose-dependent inhibitory effect on MLV infection that was clearly apparent at all levels of input inocula. For example, for inocula containing 300 relative units of RT, the presence of CEM15 during virus production caused about a ten-fold reduction of infectivity. Thus, the anti-retroviral property of CEM15 can be extended to the simple (non-vif encoding) retrovirus, MLV. Similar data were obtained in parallel challenges of N-3T3 cells (data not shown).

Genetic evidence indicating that CEM15 can trigger deamination of dC to dU in DNA led to the hypothesis that retroviral reverse transcripts (cDNAs) are the physiological substrate for the deamination, and the conclusion that this appeared to be a crucial step in the CEM15/APOBEC3G-mediated suppression of viral infection (Harris et al., Nat. Immunol. (2003 in press)). Therefore, a biochemical assay was used to verify that purified recombinant CEM15 was able to directly deaminate dC in DNA.

For the DNA deamination assay, a non-epitope His₈-tagged human recombinant CEM15 was expressed in E. coli as previously described (Harris et al., Mol. Cell 10: 1247-1253 (2002); Petersen-Mahrt et al., J. Biol. Chem. 278:19583-19586 (2003)) for APOBEC1, and then purified on Ni-ATA-Sepharose (Qiagen). Subcloning was done using PCR into pIRES-Zeo (Clontech). DNA deamination was monitored using the UDG-based assay with biotinylated oligonucleotides SPM167 and SPM168 (Petersen-Mahrt et al., 2003).

It was found, as with the APOBEC family members, AID (Bransteitter et al., Proc. Natl. Acad. Sci. U.S.A. 100:4102-4107 (2003); Chaudhuri et al., Nature 422:726-730 (2003)) and APOBEC1, that CEM15 was, indeed, able to deaminate dC->dU in single-stranded DNA (FIG. 6B).

### Example 4

### Retroviral cDNA is the Physiological Substrate for the DNA Deaminating Activity of CEM15

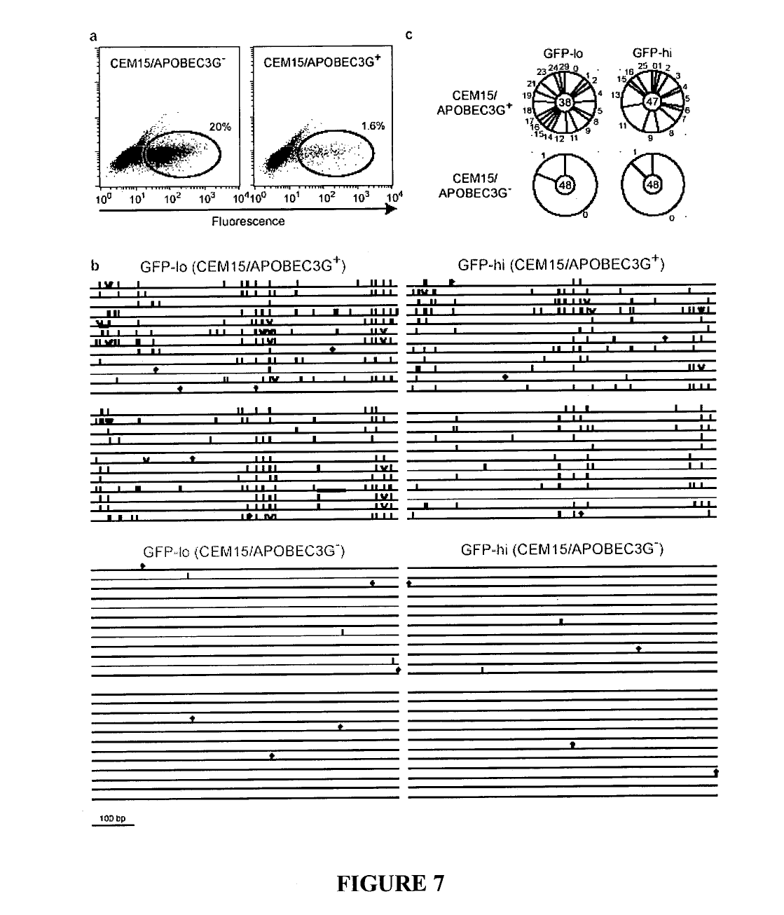

To begin to assess whether retroviral cDNA might be the physiological substrate for the DNA deaminating activity of CEM15, a GFP-encoding MLV was again produced as above in 293T cells that stably express (or do not express) human CEM15 in the producer cell. As in the YFP experiments above, expression of CEM15 in the producer cell resulted in a substantial inhibitory effect on MLV infection, as judged by diminished fluorescence of the reporter cells 48 hours after being challenged with the MLV-GFP virions (FIG. 7A). MLVGFP was the gift of F. Randow (Cambridge, U.K.).

Reasoning that dC->dU deamination of the retroviral reverse transcripts could give rise to inactivating mutations of the GFP gene, it was hypothesized that some of the GFP-negative target cells could, nevertheless, have been infected, but that the GFP gene could have been rendered non-functional by CEM15-induced mutations. As a result, target cells that were negative/lo for GFP expression (as well as their GFP-hi counterparts) were, therefore, isolated by flow cytometry, and the retroviral DNA present in the cell lysates was amplified by PCR, cloned and sequenced.

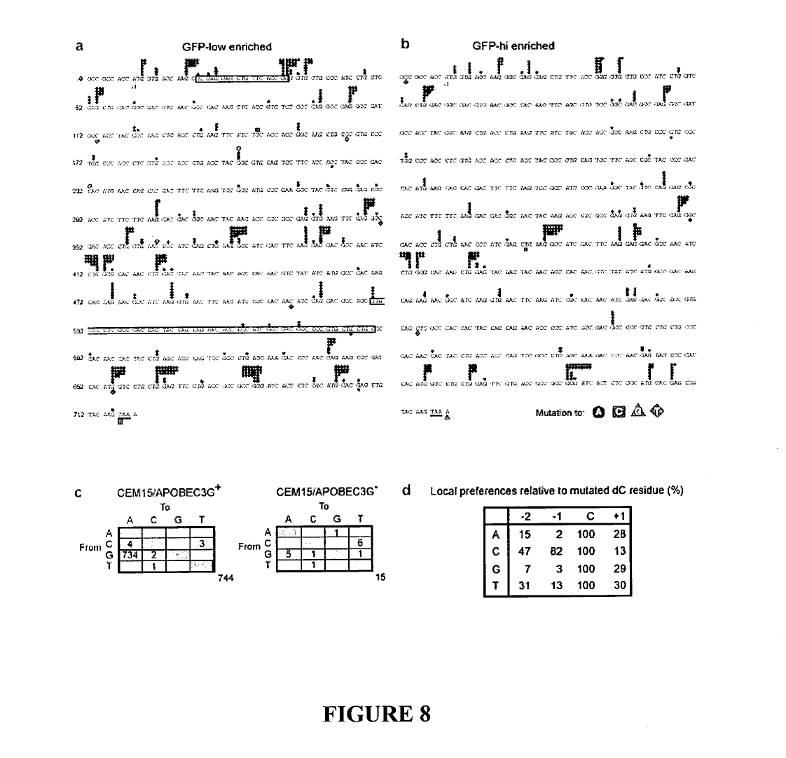

A comparison of the GFP gene sequences recovered from GFP-lo cells revealed a striking level of plus-strand G->A transition mutation, but only when the input virus was derived from CEM15-expressing cells (FIGS. 7B, 7C and FIG. 8). Two independently derived CEM15+clones (and two independent controls) were analyzed for each experiment.

For the retroviral DNA sequence analyses, DNA (Qiagen) was purified from populations of GFP-lo and GFP-hi cells isolated by flow cytometry (DakoCytomation, Fort Collins, Colo.). Gates were established such that only the dullest (GFP-lo) and brightest (GFP-hi) 1% of all cells were collected. A 98% enrichment of the desired populations was achieved as judged by re-analysis using a different FACS. DNA was subject to high-fidelity PCR (Pfu Turbo DNA polymerase, Stratagene, La Jolla, Calif.) using oligonucleotides 5′-TAGACGGCATCGCAGCTTGGA (SEQID NO:11) and 5′-CTGGTGATATTGTTGAGTCA (SEQID NO:12), gel purified and cloned using internal Hind III and Not I restriction sites. The resulting 730 bp fragment contained the entire GFP coding sequence (+1 to 721, FIG. 8). Sequences bearing an identical set of mutations were counted only once. Although it is possible that some clones are dynastically related (causing over-counting of some mutations), such a possibility is highly unlikely to introduce major skewing since all experiments were performed with a replication-deficient virus, and the same distribution of hotspots was identified in independent sets of sequences using virus produced from independent CEM15-expressing producer cells.

FIG. 7B provides profiles of the relative positions of the G->A transition mutations apparent in 12 representative 730 bp GFP sequences. Each pair of panels (lo and hi) in FIG. 7B represents sequences recovered from 293T target cells, but the viral stocks used in each experiment (each panel pair) were derived from independent clones either expressing or not expressing CEM15. A total of 435 G->A transitions were found to be distributed over 86 guanosine residues in 38 sequences (27,740 bp). This resulted in a mutation frequency of >1.5 mutations per 100 bases.

In contrast, only three separate G->A transitions were found in 47 sequences (34,310 bp) derived from the GFP-lo sorted reporter cells that had been challenged with MLV derived from a non-CEM15 expressing producer cell line. However, both sequence cohorts contained six other base substitution mutations (a total of 20 single nucleotide substitutions) likely attributable to RT or PCR errors, although two deletions were also detected in the G->A transition-littered sequences.

The level of mutation was so high (averaging 11 mutations per virus-derived 730-nucleotide GFP gene sequenced) that it was anticipated that mutations would also be evident in cDNAs obtained from GFP-positive target cells—which was, indeed, found to be the case. A total of 299 G->A transition mutations were found distributed over 47 retroviral cDNAs obtained from the GFP-hi cells that were sorted from the population challenged with MLV-GFP produced from CEM15-expressing cells (FIGS. 7B, 7C and FIG. 8). Thus, even with enrichment for the brightest fluorescing cells, 91% (43/47) of the amplified sequences harbored mutations yielding a frequency of 0.9 mutations per 100 bases.

The pie charts in FIG. 7C depict the proportion of MLV sequences carrying the indicated number of mutations within the sequenced 730 bp interval. Thus, they provide comparisons of the extent of GFP mutation in the different samples.

The mutational onslaught experienced by the GFP gene generates some obviously nonfunctional variants (FIG. 8). Although, these were especially apparent in the GFP-lo populations (FIG. 8A), probable non-functional variants were still evident in cDNAs obtained from the GFP-hi population (FIG. 8B)—presumably reflecting multiple infections of individual cells. Furthermore, it is predicted that some of the mutational variants have altered properties, such as enhanced or altered spectral properties.

The dramatic mutation of retroviral cDNA seen in the present invention is strictly dependent on CEM15. Mutated GFP genes were rarely obtained from target cells infected with virus produced in the absence of CEM15. Specifically, a total of only 9 and 6 mutations were observed in 47 and 48 sequences from the GFP-lo and GFP-hi sequences respectively, resulting in an overall frequency of only 0.012 mutations per 100 bases (FIG. 7B and FIG. 8). This background mutation differed from CEM15-triggered mutation not only in terms of frequency (more than 50-fold), but also in terms of pattern (not focussing on major hotspots) and base substitution types (less than half are G->A transitions).

When viruses were produced from CEM15-expressing cells, virtually all of the mutations (734 out of 744) were G->A transitions, as judged on the viral plus-strand (see FIG. 8C showing nucleotide substitution preferences for the entire set of GFP mutations detected in target cells infected with MLV-GFP that had been grown on a CEM15-expressing producer cells, in comparison to those detected in target cells infected with MLV-GFP grown on a vector-expressing producer cells). Since both genetic (Harris et al, 2002) and biochemical (FIG. 6B) evidence revealed that CEM15 deaminates dC->dU in DNA, the G->A transitions detected in the PCR-amplified GFP genes were attributed to CEM15-mediated dC->dU deamination in the retroviral minus (first)-strand cDNAs.

Interestingly, the distribution of mutations along the GFP gene was noticeably non-random. The vast majority of transition mutations attributable to deamination that targeted to dC residues, are preceded by a 5′-dT/dC-dC consensus (see FIG. 8D showing the preferred local sequence context for CEM15-mediated dC deamination in MLV-GFP). All 734 mutated positions were aligned with respect to the dC residue targeted for deamination on the minus (first)-strand cDNA, and the frequency (as a percentage) with which each of the four bases is found at adjacent positions was calculated. This specificity accords well with the context preference that was previously evident from CEM15-induced mutation in E. coli (Harris et al., 2002).

### Example 5

### CEM15 Is Transferred from Virus Producing Cells into Target Cells as a Virion Component and Its Function Is Inhibited by HIV Vif

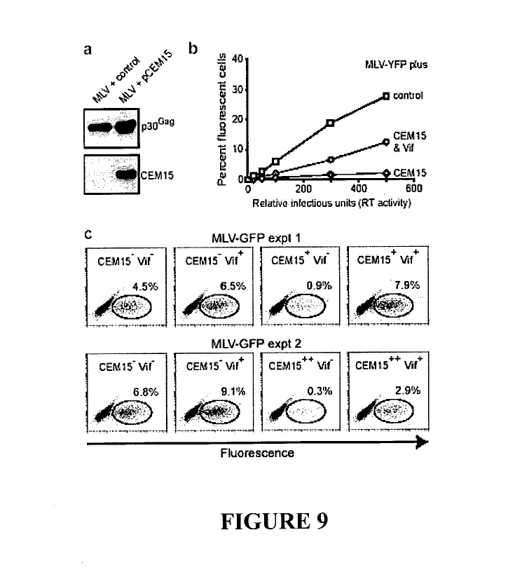

The CEM15-mediated suppression of HIV infection was thought to be mediated by CEM15 molecules that are transferred from virus producing cells into target cells as a virion component (Sheehy et al., 2002). To show that a similar mechanism could apply in the MLV system used in the present invention, viral particles (YFP-encoding MLV virions) were recovered by centrifugation at 20,000 g for 60 minutes from culture supernatants produced in the presence or absence of CEM15 as described in Example 3, (FIG. 6A). Pellets were resolved by SDS-PAGE and subjected to immunoblot analysis using either goat anti-MLV Gag antiserum (Quality Biotech, Inc., Camden, N.J.) or an anti-HA monoclonal antibody (Covance), HRP-conjugated secondary antibodies and enhanced chemiluminescence to show CEM15 (FIG. 9A). As seen in lane 2, CEM15 is indeed incorporated into the MLV virions. This presumably ensures association with reverse transcription complexes in newly infected cells and proximity to nascent cDNA substrates for the execution of dC deamination.

Nevertheless, because CEM15-mediated inhibition of HIV infection is counteracted by Vif, it was also important to test whether this held true for MLV. Co-expression of Vif in CEM15-expressing cells (either transiently or stably) was able to rescue much of the productive MLV infection as monitored by fluorescence (FIGS. 9B, 9C).

As shown in FIG. 9B, HIV Vif diminishes CEM15/APOBEC3G-mediated immunity to YFP-encoding MLV as monitored using the transient expression system (as in FIG. 6) in the presence of pCEM15:HA (3 μg) with or without pcVIF (1 μg). Plasmid pcVIF is a pBC12-based vector containing the HIV-1 IIIB vif gene as an XbaI-SalI restriction fragment, and four downstream copies of the M-PMV CTE. The infectivity of CEM15-exposed MLV-GFP was restored by expression of HIV Vif during viral stock production from 293T cells stably expressing CEM15 (FIG. 9C). The marginal restoration of infectivity in correlates with the approximately three-fold higher expression of CEM15 in this 293T-based cell line than in the individual cell line used in above. This result shows that Vif is less able to overcome the effects of high level CEM15 expression, providing a strong rationale for why overexpression is therapeutically desirable.

Thus it was necessary to answer the question of how CEM15-mediated DNA deamination of the retroviral first strand cDNA contributed to the innate immunity during normal retroviral infection in vivo. One possible answer was that the level of mutation achieved is (as may be the case in the experimental system used in the present invention) so high that it jeopardizes viral expansion. Indeed, hypermutation has been described in HIV, and is typically characterized by excessive G->A transitions (Vartanian, et al., J. Virol. 65:1779-1788 (1991); Janini, et al., J. Virol. 75:7973-7986 (2001)). However, CEM15-mediated blockade of HIV infection has been shown to be accompanied by a failure to accumulate retroviral cDNAs in target cells (Simon et al., J. Virol. 70:5297-5305 (1996)). Accordingly, it has been proposed (Harris et al., Nat. Immunol. (2003 in press)), that dC->dU deamination of retroviral first strand could, rather than simply leading to a massive accumulation of viral cDNA mutations, trigger ablation of viral infection since the presence of uracil in the first strand cDNA could recruit components of the base excision repair pathway resulting in the severance of viral replication intermediates. In other words, it may well be that, without intending to be so limited, mechanistically the CEM15-mediated dC->dU deamination in the first strand cDNA that is read out in the experimental system used in the present invention, in terms of G->A hypermutation of the plus strand of DNA, could function under physiological conditions to facilitate clearance of systemic viral infection by triggering a uracil-based excision pathway.

Such a concept of an innate nucleic acid deamination mechanism may well be applicable to other viruses, since the results described herein indicate that CEM15 carried through in virions from producer cells acts to deaminate dC->dU in retroviral minus (first)-strand cDNAs, which are the likely trigger for its antiviral effect. Hypermutation (G to A) has also been described for the hepadnavirus, hepatitis B virus (HBV), making this a likely further substrate for CEM15 (or other APOBEC family member) deamination and suppression of replication. Moreover, since APOBEC-1 edits RNA, rather than DNA, this family of proteins may target (and inhibit) both RNA- and DNA-based viruses. As a result, exploitation of the APOBEC family of cellular cytidine deaminases will have broad applicability to the development of novel anti-viral therapeutics.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. A method of inhibiting, suppressing or preventing viral infection in vertebrate cells, comprising deaminating deoxycytidine (dC) to deoxyuridine (dU) in an infecting viral nucleic acid in a dose-dependent manner by introducing a cellular nucleic acid deaminase, thereby establishing an innate cellular immune defense mechanism against infectious (non-cellular) nucleic acid.

2. The method of claim 1, wherein the vertebrate cell is selected from the group consisting of human, primate, or non-primate origin.

3. The method of claim 1, wherein nucleic acid is deoxyribonucleic acid (DNA) or ribonucleic acid (RNA).

4. The method of claim 1, wherein the nucleic acid deaminase is an APOBEC family protein.

5. The method of claim 2, wherein the APOBEC family protein is CEM15 (also known as APOBEC3G).

6. The method of claim 1, wherein the viral infection is the result of a retrovirus.

7. The method of claim 1, wherein the viral infection is HIV or MLV.

8. The method of claim 7, wherein the viral infection is HIV-1.

9. The method of claim 6, wherein said method of inhibiting, suppressing or preventing retrovirus infection in a vertebrate comprises providing an APOBEC family protein to the virus infecting cell, or infecting virions, or a combination thereof; triggering deamination of deoxycytidine (dC) to deoxyuridine (dU) in DNA of the virus in a dose-dependent manner, thereby mutating the DNA; and forming an innate immune defense mechanism against the retrovirus infection in the vertebrate species.

10. The method of claim 9, wherein the APOBEC protein is CEM15 (also known as APOBEC3G).

11. The method of claim 10, wherein the CEM15 is vif-resistant CEM15.

12. An isolated nucleic acid sequence encoding CEM15 protein or analog thereof, having the functional ability upon introduction into an retrovirus particle in a vertebrate cell to trigger deamination of deoxycytidine (dC) to deoxyuridine (dU) in DNA of the virus in a dose-dependent manner, thereby mutating the DNA and forming an innate defense mechanism against retroviral infection in said cell.

13. The isolated nucleic acid of claim 12, wherein said nucleic acid shares at least about 50% homology with SEQ ID NO: 1.

14. The isolated nucleic acid of claim 12, said nucleic acid further comprising a reporter nucleic acid covalently linked thereto.

15. A purified polypeptide comprising CEM15 protein or analog thereof, which is the target of Vif function during normal retrovirus replication, and having the functional ability upon introduction into a retrovirus infected cell, or retrovirus virion, or combination thereof in a vertebrate cell to trigger deamination of deoxycytidine (dC) to deoxyuridine (dU) in DNA of the virus in a dose-dependent manner, thereby mutating the DNA and forming an innate immune defense mechanism against said retroviral infection in said cell.

16. The isolated polypeptide of claim 15, wherein said polypeptide shares at least about 50% homology with SEQ ID NO: 2.

17. A retrovirus infected cell comprising the isolated nucleic acid of claim 12.

18. A recombinant cell comprising the isolated nucleic acid of claim 12.

19. The cell of claim 17, wherein said cell is selected from the group consisting of a prokaryotic cell and a eukaryotic cell.

20. The cell of claim 19, wherein CEM15 is expressed in said cell.

21. The cell of claim 19, wherein said nucleic acid shares at least about 50% homology with SEQ ID NO: 1.

22. The cell of claim 19, wherein the expressed CEM15 is a Vif-resistant form of CEM15 in the CEM15 expressing cell.

23. A vector comprising the isolated nucleic acid of claim 12.

24. A virion comprising the isolated nucleic acid of claim 12.

25. The virion of claim 24, wherein said nucleic acid shares at least about 50% homology with SEQ ID NO: 1.

26. A vertebrate cell comprising the virion of claim 24.

27. A vertebrate cell comprising the virion of claim 25.

28. A therapeutic pharmaceutical formulation for inhibiting, suppressing or preventing viral infection in a vertebrate, comprising an isolated nucleic acid sequence in a pharmaceutically acceptable carrier, said nucleic acid encoding a cellular nucleic acid deaminase protein or analog thereof, having the functional ability upon introduction into a virus infected cell, or virus virion, or combination thereof in a vertebrate cell to trigger deamination of deoxycytidine (dC) to deoxyuridine (dU) in DNA of the virus in a dosedependent manner, thereby mutating the DNA and forming an innate defense mechanism against viral infection in the cell.

29. The therapeutic pharmaceutical formulation of claim 28, wherein the nucleic acid deaminase is an APOBEC family protein.

30. The therapeutic pharmaceutical formulation of claim 29, wherein the APOBEC family protein is CEM15 (also known as APOBEC3G).

31. The therapeutic pharmaceutical formulation of claim 28, wherein said nucleic acid shares at least about 50% homology with SEQ ID NO: 1.

32. The therapeutic pharmaceutical formulation of claim 28, wherein the virus is a retrovirus, the virion is a retrovirus virion, and the infection is retroviral.

33. The therapeutic pharmaceutical formulation of claim 32, wherein the nucleic acid deaminase is an APOBEC family protein.

34. The therapeutic pharmaceutical formulation of claim 33, wherein the APOBEC family protein is CEM15 (also known as APOBEC3G).

35. The therapeutic pharmaceutical formulation of claim 32, wherein said nucleic acid shares at least about 50% homology with SEQ ID NO: 1.

36. A therapeutic pharmaceutical formulation for inhibiting, suppressing or preventing viral infection in a vertebrate comprising a purified polypeptide, comprising a cellular nucleic acid deaminase or analog thereof, in a pharmaceutically acceptable carrier, wherein said polypeptide is the target of Vif function during normal HIV-1 replication, and having the functional ability upon introduction into a retrovirus infected cell, or a retrovirus virion, or a combination thereof in a vertebrate cell to trigger deamination of deoxycytidine (dC) to deoxyuridine (dU) in DNA of the virus in a dose-dependent manner, thereby mutating the DNA and forming an innate immune defense mechanism against retroviral infection in said cell.

37. The therapeutic pharmaceutical formulation of claim 36, wherein the nucleic acid deaminase is an APOBEC family protein.

38. The therapeutic pharmaceutical formulation of claim 37, wherein the APOBEC family protein is CEM15 (also known as APOBEC3G).

39. The therapeutic pharmaceutical formulation of claim 36, wherein said polypeptide shares at least about 50% homology with SEQ ID NO: 2.

40. The therapeutic pharmaceutical formulation of claim 36, wherein said polypeptide is a Vif-resistant form of CEM15.

41. A method for inhibiting, suppressing or preventing viral infection in a vertebrate infected by a viral infection or subject to such infection, comprising administering to said vertebrate an effective amount of, or effecting expression or overexpression in said vertebrate, the therapeutic pharmaceutical formulation of claim 28.

42. The method of claim 41, wherein inhibiting, suppressing or preventing viral infection in a vertebrate infected by a viral infection or subject to such infection is achieved in conjunction with other therapeutic agents.

43. The method of claim 41, wherein inhibiting, suppressing or preventing viral infection in a vertebrate infected by a viral infection or subject to such infection is achieved by gene therapy.

44. A method for inhibiting, suppressing or preventing viral infection in a vertebrate infected by a viral infection or subject to such infection, comprising administering to said vertebrate an effective amount of, or effecting expression or overexpression in said vertebrate, the therapeutic pharmaceutical formulation of claim 29.

45. A method for inhibiting, suppressing or preventing viral infection in a vertebrate infected by a viral infection or subject to such infection, comprising administering to said vertebrate an effective amount of, or effecting expression or overexpression in said vertebrate, the therapeutic pharmaceutical formulation of claim 30.

46. The method of claim 36, wherein inhibiting, suppressing or preventing viral infection in a vertebrate infected by a viral infection or subject to such infection is achieved in conjunction with other therapeutic agents.

47. A method for inhibiting, suppressing or preventing viral infection in a vertebrate infected by a viral infection or subject to such infection, comprising administering to said vertebrate an effective amount of, or effecting expression or overexpression in said vertebrate, the therapeutic pharmaceutical formulation of claim 31.

48. A method for inhibiting, suppressing or preventing viral infection in a vertebrate infected by a viral infection or subject to such infection, comprising administering to said vertebrate an effective amount of, or effecting expression or overexpression in said vertebrate, the therapeutic pharmaceutical formulation of claim 32.

49. An assay for monitoring the innate immune response produced in accordance with claim 1 in a MLV-based system, comprising contacting a test cell with a cellular nucleic acid deaminase or placed in the presence of a cellular nucleic acid deaminase expressing cell, such that virus present in the test cell at the time of contact, or after such contact is unable to replicate, and deamination of deoxycytidine (dC) to deoxyuridine (dU) is triggered in DNA of the virus in a dose-dependent manner.

50. The assay according to claim 49, wherein the nucleic acid deaminase is CEM15 or an analog thereof.

51. A method of random mutagenesis of genes or genetic elements in a test cell, comprising contacting said test cell with a cellular nucleic acid deaminase or placed said cell in the presence of a cellular nucleic acid deaminase expressing cell, such that the DNA of the virus present in the test cell at the time of contact, or after such contact, is subjected to deamination of deoxycytidine (dC) to deoxyuridine (dU) in a dose-dependent manner, thereby mutating the nucleic acid of the virus.
